(19) United States
(12) Patent Application Publication
(10) Pub. No.: US 2026/0366050 A1
(43) Pub. Date: Nov. 19, 2026

(54) DOUBLE FERMENTATION OF BIOMASS FOR PRODUCTION OF LACTIC ACID

(71) Applicant: William A. Hastings, Wasilla, AK (US)

(72) Inventor: William A. Hastings, Wasilla, AK (US)

(21) Appl. No.: 19/671,931

(22) Filed: May 8, 2026

Related U.S. Application Data
(60) Provisional application No. 63/803,188, filed on May 9, 2025.

(51) Int. Cl. C12P 7/56

(52) U.S. Cl. CPC C12P 7/56; C12P 2203/00

(57) ABSTRACT

Disclosed are methods for fermenting fresh, raw, plant materials using a multistep fermentation process. Plant materials of a larger size (e.g., around 2 inches or more in length and thinly cut/sliced in size to increase surface area) are fermented in a first step to produce a fermentate. The first-step fermentate is reduced in size (e.g., ground, blended, slurried) and fermented in a second step. Also disclosed are products of the fermentation methods.

All patents, patent applications and publications cited herein are hereby incorporated by reference in their entirety. The disclosures of these publications in their entireties are hereby incorporated by reference into this application in order to more fully describe the state of the art.

U.S. patent application Ser. No. 19/083,054, filed Mar. 18, 2025, published as U.S. 2025/0313517 A1 on Oct. 9, 2025, and titled “Carbon-Negative Fertilizer and Methods for Making” is incorporated herein by reference.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### CROSS-REFERENCE TO RELATED APPLICATIONS

This application claims the benefit and priority to U.S. Provisional Application No. 63/803,188, filed on May 9, 2025, the entire contents of which are incorporated herein by reference.

### BACKGROUND

Industrial lactic acid has been determined to be, among other things, a very useful raw material for the production of polylactic acid (PLA)-a bio polymer used to make biodegradable plastics. Lactic acid is also used in a myriad of other industrial applications and is generally very expensive to produce. In the current art, industrial-grade lactic acid can be produced either through chemical processes or fermentation. Chemical processing is overall more expensive than fermentation methods and results in an inferior grade of lactic acid. Therefore, most of the current processes/methods used in the world today to produce lactic acid are via biological fermentation. Yet, the lactic acid produced via biological methods remains an expensive end product. There has been significant research and development devoted to reducing the overall costs associated with fermentation methods, in order to make fermentative-based lactic acid an economical endeavor.

### SUMMARY

Disclosed herein are methods to produce lactic acid, on an industrial scale, by fermenting fresh, raw, unprocessed plants or, fresh, raw, unprocessed plant material(s) (e.g., fresh, raw, unprocessed terrestrial or aquatic materials), via lactic acid-producing microorganism(s) (e.g., lactic acid bacteria), into a shelf-stable, nutrient-rich material. In some embodiments, the plants and/or plant materials have a moisture level at or above 50% dry matter. In some embodiments, these plant and/or plant materials are chopped, chipped or shredded into material that is 2 inches or greater in size. Wilting of material is allowable but increases the likelihood of attack by undesirable microorganisms. In some embodiments, herein called the Double Fermentation Embodiment, a first step of the method produces a product that has been fermented but has not been “fermented to completion” (i.e. fermented to the point where the biomass has been exhausted of carbohydrates and protein by the fermentation of lactic acid microorganisms), herein called the First Fermentation Step. The fermented product of the First Fermentation Step can be stored for an indefinite time and then used to continue the fermentation to completion (the Second Fermentation Step) at a chosen time.

In some embodiments, the First Fermentation Step ends after accessible sugars are consumed, if the pH falls to a level inhospitable to microbes (all types of microbes), the temperature drops below levels conducive to all microbiological activity or a combination of all three (nutrient availability, pH, temperature). These factors can, either individually or in combination, create a pause in the fermentation. In some embodiments, the fermenting microbes can become “dormant.” The product of the First Fermentation Step can be processed to make additional fermentable material available for fermentation in the Second Fermentation Step.

As above, material that results from the First Fermentation Step can be processed into smaller pieces prior (<2″ in size) to the Second Fermentation Step. By grinding, blending and the like, the material into a fine slurry, for example, the surface area of the material created by the First Fermentation Step increases, along with the pH (due to released protein buffers), making accessible and releasing the preserved sugars, proteins, and other nutrients for the Second Fermentation Step. The Second Fermentation Step can further convert the nutrient-rich material into lactic acid for industrial purposes (i.e. the “Double Fermentation”). Use of this processed material in the Second Fermentation Step can result in further fermentation, and in further production of lactic acid.

The product of the First Fermentation Step, therefore, can be fermented further (e.g., to completion) during the Second Fermentation Step.

In embodiments, the Second Fermentation Step of the nutrient-rich, previously fermented material does not require addition of nutrients or buffering chemicals for the fermentation to initiate and occur. In some embodiments, buffering chemicals and/or nutrients can be added to sustain the Second Fermentation Step, such that increased amounts of lactic acid can be produced.

In other embodiments, a Single Fermentation Embodiment (in contrast to the Double Fermentation Embodiment described above) is performed. In the Single Fermentation Embodiment, the starting material for the fermentation is ground, blended and the like, to a size less than about two inches before the fermentation is performed (e.g., to expose additional microbe-accessible material). In some examples of the Single Fermentation Embodiment, raw, fresh, unprocessed plants/plant materials (terrestrial or aquatic) are ground, blended and the like, and fermented to completion without a Second Fermentation Step. Again, lactic acid is produced. The Single Fermentation Embodiment method does not require addition of nutrient or buffering chemicals to initiate the fermentation but buffering chemicals and/or nutrients may be added to sustain the fermentation and/or to produce maximum lactic acid production.

In some embodiments, the Double Fermentation Embodiment (First Fermentation Step) is used as a way to preserve and store raw plant materials and raw plant materials (terrestrial or aquatic) as substrates/feedstocks for follow-on fermentation (Second Fermentation Step) and processing into industrial lactic acid. This includes, but is not limited to, easily spoilable plants and parts of plants such as turnips, radishes, beets, their roots and especially their leaves, plants considered pests for agriculture (e.g. “weeds”), and aquatic plants such as kelp or freshwater algae.

In some embodiments, the inventions disclosed herein focus on improvements to the pretreatment phase of industrial lactic acid production. Lactic acid production relies on the current art of post-treatment to process the fermented biomasses described herein into industrial-grade lactic acid (e.g., to isolate and refine lactic acid from the fermentate or fermented biomass).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A-B illustrates an example of Galeopsis tetrahit aka hemp nettle (a State of Alaska designated “noxious” weed) fermentate with (B) and without (A) sugar inoculant.

FIG. 2A-B illustrates an example of Chamerion angustifolium (fireweed) fermentate, with (B) and without (A) sugar inoculant.

FIGS. 3A and 3B show Double Fermentation Samples for certified testing.

FIGS. 4A and 4B show Double Fermentation Samples for daily pH and gas buildup monitoring.

FIG. 5 shows steps currently used for converting different feedstocks into industrial-grade lactic acid

FIG. 6 shows a schematic of the steps for the Double and Single Fermentation Embodiments.

FIG. 7A shows the major steps of the Double Fermentation Embodiment, comparing the current art and vs. the invention used for converting fresh fermented biomass (i.e. fresh fermented Double Fermentation biomass in this case).

FIG. 7B shows the major steps of the Single Fermentation Embodiment comparing the current art and vs. the invention used for converting fresh fermented biomass (i.e. fresh fermented Single Fermentation biomass in this case).

FIG. 8 illustrates example probiotic culture count of colony forming units.

FIG. 9 illustrates example DNA sequencing and identification of colony forming units (Lactobacillus) as shown in FIG. 8.

FIG. 10A-C illustrates examples of harvested and shredded turnips.

FIG. 11A-B illustrates examples of turnip fermentate.

### DETAILED DESCRIPTION

### Introduction

Disclosed are methods to produce lactic acid by fermenting fresh, raw plants or fresh raw plant materials, be they terrestrial or fresh aquatic or salt aquatic via lactic acid-producing microorganisms, into shelf-stable, nutrient-rich material that contains lactic acid. In some embodiments, a Double Fermentation Embodiment is disclosed that ferments plants or plant materials for a time and then pauses the fermentation (the First Fermentation Step) before fermentation goes to completion. In some embodiments, the fermentation can be paused by placing the materials under conditions that do not favor further fermentation (e.g., cooler temperature, low pH). In some embodiments, the starting material for the First Fermentation Step can be of a size (e.g., 2 inches in length or so; cut/sliced to increase surface area) such that some of the material is not microbe accessible and the fermentation slows or stops when the microbe-accessible material has been completely fermented, or the pH drops to inhospitable levels when the microbe accessible plant protein buffers have been exhausted, or the accessible sugars to the microbes have been exhausted.

For example, in some embodiments of the Double Fermentation Embodiment, fresh, raw, unprocessed material, at moisture levels above 50% dry matter (with no upper limit of moisture content), can be cut, chipped or shredded into pieces of about two inches in size. These pieces are packed to reduce air pockets and can undergo anaerobic lactic acid fermentation (First Fermentation Step) and create a material called “fresh fermented biomass”. When this first fermentation step has stopped or been interrupted, the fresh fermented biomass consists of lactic acid, preserved sugars, and preserved plant protein buffers. The fresh fermented biomass is now storable and shelf-stable. At a time of our choosing, the fresh fermented biomass is then made into smaller pieces by, for example, mincing, grinding crushing, pulverizing or powdering the fermentate. This reduced-size fresh fermented biomass begins the Second Fermentation Step whereby the preserved sugars and the preserved plant protein buffers are released, allowing the dormant lactic acid microorganisms to restart fermentation. Fermentation will continue until all remaining sugars are consumed. The “fresh fermented biomass” has been “fermented to completion” once all sugars have been converted into lactic acid. If plant protein buffers are consumed before plant sugars have been exhausted, then the addition of outside buffers, and possibly additional nutrients or enzymes, are used to “ferment to completion”.

The pause at the end of the First Fermentation Step allows for storage of the material (aka “fresh fermented biomass”), which can be advantageous because the fermented material eventually must go to processing facility to extract the lactic acid. The pause in the fermentation in the disclosed method provides for more flexible scheduling in transporting the fresh fermented biomass to the processing facility (e.g., the Second Fermentation Step can be initiated at a time such that its completion, resulting in completely fermented material aka “fermented to completion”), occurs at a time convenient for transport to the processing facility. The pause also provides for some processing of the fermentate. In some embodiments, the “fresh fermented biomass” produced from what is called the First Fermentation Step can be ground, blended and the like, to reduce the size of the particles in the fermentate (e.g., to less than two inches in size) so that any unfermented material is made available to lactic acid producing bacteria in the resumed fermentation, called the Second Fermentation Step.

In some embodiments, a Single Fermentation Embodiment is disclosed in which the fresh, raw, unprocessed plants or plant materials are ground, blended and the like, to a small size (e.g., less than 2 inches or so in length and thinly cut/sliced to increase surface area) prior to the fermentation.

For example, in the Single Fermentation Embodiment, raw plants or plant materials can be processed into small pieces at the outset (e.g., mincing, grinding crushing, pulverizing or powdering). The small-sized plant material is then fermented to completion and lactic acid is purified from the fermentate.

### DEFINITIONS

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Herein, “grinding”, “blending” and the like, in relation to the plants disclosed herein, can refer to an act whereby whole plants or parts thereof, be them terrestrial or aquatic, are made into fine pieces, less than 2″ in size for example, or a slurry. These processes increase surface area of plant material, can improve ability and speed of bacteria to colonize and metabolize (i.e. ferment) the raw plant material, create a generally uniform size of material which can aid expunging of air/oxygen, release confined sugars contained within plant cell walls, and release plant protein which can buffer the biomass solution and provide for further fermentation by lactic acid bacteria.

Herein, “chopping”, “chipping”, “shredded” or the like, in relation to the plants and plant materials, be them terrestrial or aquatic, disclosed herein, can refer to an act whereby the whole plant or parts thereof, be them terrestrial or aquatic, are made into pieces around 2″ or greater in size. These mechanical processes expose a relatively small amount of surface area of the plant material and the sugars contained within; enough surface area and sugars to support a rapid fermentation by lactic acid producing microorganisms but small enough whereby the fermentation also rapidly stops, preserving the bulk of the original nutrition from the original biomass.

Herein, “compacting” in relation to the plants or chipped/shredded plants disclosed herein can refer to applying direct pressure, via mechanical means, or indirect pressure, via weight of the material, such that the plants are packed closer together, to remove air pockets and expunge as much oxygen as possible, to enhance anaerobic fermentation by lactic acid microorganisms.

Herein, “Double Fermentation Embodiment” can refer to a fermentation method whereby the raw substrates on the field can be preserved by partial lactic acid fermentation and stored indefinitely, until ready for transporting to a lactic acid production facility. The substrates are chopped, shredded, and/or chipped (e.g., 2 inches or greater in size) and then fermented by lactic acid producing microorganisms, creating “fresh fermented biomass”. When needed, the “fresh fermented biomass” material is ground or blended into finer particles (e.g., less than 2″ in length) or into a slurry, to increase the surface area, and to restart the lactic acid fermentation. In some embodiments, this processing can occur at an industrial lactic acid production facility.

Herein, “effluent” can refer to liquids which are produced and accumulate (if provisions for draining the effluent are not in place during fermentation) during a fermentation process. The effluent liquids can be rich in lactic acid, as well as plant-available nitrogen, phosphorus, and/or potassium.

Herein, “feedstocks” can refer to either (1) starches, either processed (e.g., corn starch, rice starch, etc.) or raw (e.g., raw, dry grains) meant for the generation of industrial lactic acid, (2) lignocellulosic agricultural waste materials (e.g. coffee cut stems, corn cobs, sugarcane bagasse, waste paper sludge, etc.) meant for the generation of industrial lactic acid, or (3) raw plants or plant materials, be they terrestrial or aquatic plants (fresh or saltwater) or parts of plants which, when fermented, contain lactic acid. In some embodiments, plant material that has not been fermented to completion or which, upon processing to a smaller size can be further fermented (e.g., product of the First Fermentation Step of the Double Fermentation Embodiment), are considered feedstocks. Herein, these feedstocks can be fermented and/or processed to obtain lactic acid.

Herein, “fermentate” can refer to a plant mixture that is undergoing or has completed fermentation.

Herein, the term “fermentation” can refer to an anaerobic process by which glucose is broken down. ATP and other compounds (e.g., lactic acid) are produced. Herein, the fermentate (e.g., the plant mixture that undergoes or has undergone fermentation) may also contain microbes that produce ATP via aerobic processes, as long as there is some anaerobic catabolismin the mixture. Herein, fermentation involves bacteria that produce lactic acid. In some embodiments, the bacteria are Lactobacillus.

Herein, “fermented-to-completion” or “complete fermentation” refers to fermented material that has little or no material left that can be fermented by lactic acid-producing bacteria. An example of “fermented-to-completion” is the stage after lactic acid microorganisms have ceased producing lactic acid, and the lactate salts formed due to the addition of inorganic buffers (such as calcium hydroxide) must be dissolved back into lactic acid through the addition of inorganic acids (such as sulfuric acid). In some embodiments, material (e.g. fresh fermented biomass) that has been fermented (e.g., plant pieces or 2 inches or greater in length and thinly cut/sliced to increase surface area) can be fermented to completion, by grounding or blending to a smaller size such that additional plant material becomes microbe accessible and fermentation can resume.

Herein, “fresh fermented biomass” is fermented biomass (e.g., fermentate), which is originally fresh, raw plants, terrestrial or aquatic (salt or fresh water), or plant materials, terrestrial or aquatic (salt or freshwater), which are fermented by the Double Fermentation Embodiment or the Single Fermentation Embodiment. “Fresh fermented biomass” either comes from raw plants/plant materials directly from harvest that have been chopped/chipped/shredded and fermented (first and second steps of Double Fermentation Embodiment) or comes from raw plants/plant materials directly from harvest that have been chopped/chipped/shredded/ground/blended directly into a slurry and fermented (Single Fermentation Embodiment).

In some embodiments, the original fresh raw materials needed to create “fresh fermented biomass” are at least 50% moisture level by dry matter or greater, with no upper limit to moisture level. The fresh raw materials can undergo wilting prior to processing into “Fresh Fermented Biomass” but the longer they are allowed to wilt, the greater the likelihood of attack and spoilage of the fresh, raw plant materials by undesirable microbes.

Herein, “harvesting” refers to removing plants from the field (or aquatic environment).

Herein, “homofermentative” in relation to lactic acid producing microorganisms, refers to bacteria that do not produce CO₂ or ethanol during fermentation, and generate higher concentrations of lactic acid than heterofermentative bacteria.

Herein, “heterofermentative” in relation to lactic acid producing microorganisms, refers to bacteria that produce CO₂ during fermentation and less lactic acid concentrations than homofermentative microorganisms.

Herein, “industrial-grade lactic acid” refers to lactic acid produced from microorganisms for the express purpose and use for industrial applications to include use(s) as a solvent, biopolymer, food preservative, etc.

Herein, “inoculant” refers to a culture of microorganisms (e.g., bacteria) that can be added, for example, to a plant at the beginning or during a fermentation. Herein, “inoculant” can also refer to glucose, sucrose, or fructose (i.e. simple sugars), possibly dissolved in water, that can be added to the chopped/chipped/shredded plants at the beginning or during a fermentation. Herein, inoculants that are added to a plant at the beginning of a fermentation can be called a “starter culture” or a “sugar source”. The type of microorganism inoculant (or natural microorganisms present on the surface of the fresh raw material) determines the type of lactic acid produced (D-lactic acid or L-lactic acid).

Herein, “lactic acid bacteria” or “LAB” refer to an order to gram-positive bacteria. These bacteria can produce lactic acid as a result of carbohydrate fermentation.

Herein, “PLA” refers to polylactic acid, a desired end-product of industrial lactic acid fermentation.

Herein, “preserved” means to keep from spoiling or, from the Cambridge Dictionary means to keep something as it is, especially in order to prevent it from decaying or being damaged or destroyed or, in the case of food, to treat food in a particular way so that it can be kept for a long time without going bad.

Herein, “pretreatment” refers to those techniques meant to treat starches or lignocellulosic materials, so as to be made ready for enzymatic hydrolysis for fermentation by lactic acid producing microorganisms. “Pretreatment” can include some or all of the following: mechanically grinding, shredding, blending of substrates; chemical reduction (via use of acids, alkali's oxidizing agents, and/or organic solvents); physiochemical (via use of steam explosion, autohydrolysis, hydrothermolysis, and/or wet oxidation); biological methods (via use of enzymes). Double and Single Fermentation methods, when using fresh fermented biomass, avoid almost all “pretreatment” techniques and activities required of starches and lignocellulosic biomass materials.

Herein, “restricted fermentation” refers to fermentation that is prolonged, for example by the buffering capacity of protein. In some embodiments, the high protein levels prevent a rapid drop in pH and thus, prevent rapid cessation of lactic acid production by lactic acid producing microorganisms.

Herein, “the Single Fermentation Embodiment” refers to a fermentation method whereby the raw substrates/feedstocks are taken directly from the field and chopped, chipped, shredded, and ground into fine particles (smaller than 2″) and/or slurry, to increase the surface area, and to ferment quickly and completely for the generation of industrial lactic acid. The material resulting from the Single Fermentation Embodiment is called “fresh fermented biomass”. These materials are generally not stored.

Herein, “size” in reference to raw substrates/feedstocks, can be of a large enough size such that, when material of this size is subjected to the First Fermentation Step of the Double Fermentation Embodiment, fermentation will eventually slow and perhaps stop because not all substances that could be fermented were not microbe accessible in material of this size (e.g., there is fermentable material remaining but it cannot be accessed by fermenting microbes). When slowing or stoppage of fermentation occurs under these circumstances, processing the fermented material to a smaller size can make the inaccessible material microbe accessible and fermentation can occur, as it does in the Second Fermentation Step of the Double Fermentation Embodiment. Although the starting material for the First Fermentation Step of the Double Fermentation Embodiment is referred to herein as 2 inches or greater in size, it can be smaller as long as some of the material remains at the end of the First Fermentation Step. In some embodiments, the starting material for the First Fermentation Step of the Double Fermentation Embodiment can be about 1.9, 1.8, 1.7, 1.6, 1.5, 1.4, 1.3, 1.2, 1.1, 1.0, 0.9, 0.8, 0.7, 0.6, or 0.5 inches in size.

Also, in some embodiments, the size of i) the starting, partially fermented material used in the Second Fermentation Step of the Double Fermentation Embodiment, or ii) the unfermented material used in the Single Fermentation Embodiment, is referred to herein as less than 2 inches in length, and thinly cut/sliced in size to increase surface area. Herein, this material is of a size that most or all of the material is microbe accessible, and fermentation will stop when the material is fermented (e.g., because of exhaustion of fermentable material). In some embodiments, further processing this material to a smaller size does not result in a restarting of fermentation.

Herein, “vented” in reference to an airtight container in which a fermentation is occurring, refers to opening the container to release gas.

### Feedstocks for Making Lactic Acid

Regarding substrates for lactic acid production, a first feedstock in the current art includes starches, such as processed corn starch or rice starch, as well as raw starches such as potato or corn grain, are carbohydrate-dense substrates but have several drawbacks as candidate substrate(s): (1) starches usually compete with the human and animal food supply and thus, they are more expensive, (2) processed starches are usually very low in protein, certain vitamins, and minerals-all of which are required by lactic acid bacteria for their metabolism, (3) starches (raw or processed) are harder for lactic acid bacteria to break down into simpler sugars, which the lactic acid bacteria require.

Drawback (2) is compensated for by adding expensive nutrients into the fermentation broth (e.g., protein, vitamins, macronutrients, and the like).

Drawback (3) is addressed by adding enzymes (such as amylase) and heat to break down the starches into simpler sugars. The addition of heat has the drawback of breaking down proteins and amino acids which could otherwise be used by the bacteria. After heat treatment, protein from another source must be added back into the fermentation broth; a highly inefficient and expensive requirement.

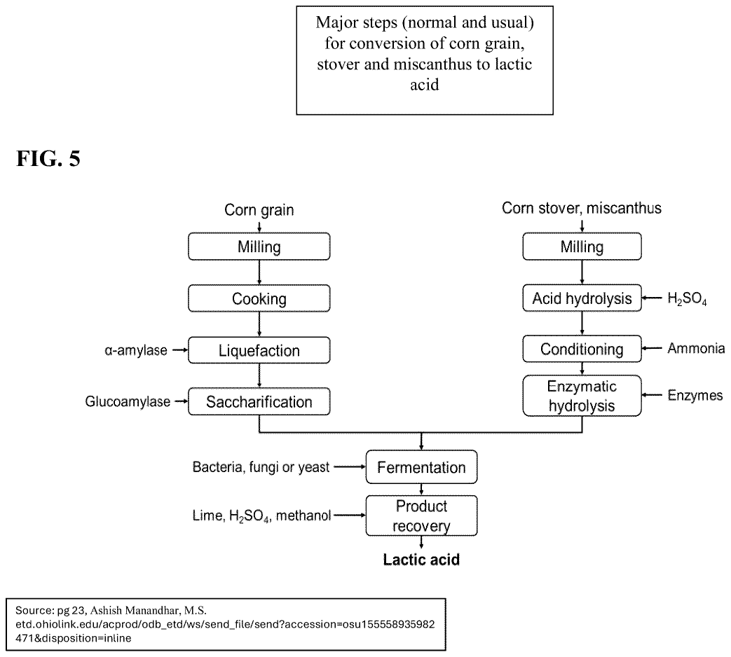

Regarding a second feedstock in the current art, lignocellulosic materials for use in fermentation, these materials can be broken into three categories: (1) hemicellulose—digestible by bacteria with assistance, (2) cellulose—digestible by bacteria with more assistance, (3) lignin—considered mostly undigestible. Lignocellulosic materials are waste byproducts of other agricultural processing (e.g. paper sludge, sugarcane bagasse, corn stover, corn cobs, etc.) and are very cheap to acquire. But using lignocellulosic isn't without costs. As waste byproducts, their valuable and easily accessible carbohydrates and/or proteins have already been extracted for use in some other product. They are then, by definition, nutrient-deprived and thus, not able to initiate and sustain lactic acid fermentation without extensive and expensive pretreatment actions. Pretreatment is necessary to break down the cell walls and access, for later fermentation, the trapped carbohydrates contained within. Pretreatment methods include applying singularly, or in combination (1) mechanically grinding the material into fine particles and/or (2) applying chemicals (e.g. alkali, dilute acids, oxidizing agents, or organic solvents) and/or (3) applying physiochemical methods (e.g. steam explosion, autohydrolysis, hydrothermolysis, or wet oxidation) (4) and/or biological methods, (e.g. enzymes). Once pretreatment has resulted in releasing the remaining carbohydrates, the material must then be augmented with whatever nutrients remain missing before fermentation may be initiated and sustained. See FIG. 5 for a general visual description of process steps and requirements for the use of both starch raw materials and lignocellulosic raw materials, in the production of industrial-grade lactic acid, in the current art.

The ideal biomass feedstock for the production of industrial-grade lactic acid is one which shares the following requirements/characteristics: (1) has all the required nutrients necessary to self-initiate and enable an “independent” “unassisted” lactic acid microorganism fermentation, (2) is low in lignin, high in soluble fibers, low in starch, and high in simple sugars, (3) is self-buffering to allow pH to resist dropping and therefore prolong the overall fermentation and production of lactic acid, (4) is storable and resists spoilage until needed/required for production of industrial-grade lactic acid, (5) has large surface area to enable lactic acid producing microorganisms easy access to nutrients but not small enough surface area whereby all nutrients are immediately consumed by the microorganisms, and (6) is a substance that is cheap, available world-wide, and does not compete with the human or animal food supply. Neither the previously described processed and concentrated starch/sugar substrates feedstocks, nor the lignocellulosic waste materials feedstocks, simultaneously share all these ideal biomass feedstock requirements, save being storable and resistant to spoilage.

The ideal biomass for lactic acid fermentation, the third feedstock described herein, are fresh, raw, unprocessed plants or parts of (terrestrial, aquatic saltwater or aquatic freshwater) plants (i.e. leaves, non-woody stems, and/or roots), as they have all the right nutrients, in the proper balance, to allow for lactic acid fermentation. But using fresh biomass has been impractical and thus dismissed as a viable option by industry because their advantage (accessible nutrients) also causes rapid attack/spoilage by undesirable microorganisms, and the costs associated with preserving this biomass is cost prohibitive and the act of preservation would remove the very nutrients, water, etc. necessary to initiate and enable an unassisted fermentation. Processing facilities would have to immediately utilize this type of material but there isn't the throughput capacity to accommodate a rapid onset of fresh material. The factory would have to be incredibly large or there would have to be an extremely high number of factories to handle the tonnage of biomass material immediately after harvest. Then, the factories would be dormant for a large part of the year, awaiting the next harvest. Added to this, transportation to these facilities would need to be extremely fast and efficient, and likely require a huge number of vehicles.

A biomass feedstock which has all of these attributes, and able to resist rapid spoilage, would be considered ideal in the art, and would represent a third, new, and heretofore unrecognized category of feedstock.

In the current art, the feedstock(s) for the production of lactic acid via fermentation are starch or sugar concentrates or lignocellulosic waste byproducts created as the result of another food processing endeavor. In the current art, buffering agents in the form of lime or ammonia (for example) are required to initiate and sustain fermentation and thus, produce high concentrations of industrial lactic acid. In the current art, fermentation is a single step to arrive at an end-state (i.e. a preserved source of animal or human nutrition or, lactic acid as an industrial chemical). In the current art, fermentation feedstocks are either starch/sugar concentrates or lignocellulosic materials, both of which require many additional inputs and steps to create a substrate lactic acid bacteria can metabolize.

The research and development efforts for reducing the costs associated with production of industrial-grade lactic acid via microorganism fermentation have been confined to these strategies and approaches: (1) find and exploit cheap, renewable substrates (i.e., “feedstocks”), and/or (2) find more effective strains of bacteria, or genetically manipulate existing strains, to improve their fermentation effectiveness, and/or (3) design new mechanical devices and/or fermentation inputs (e.g. enzymes, buffering chemicals, etc.) in order to maintain an environment conducive to lactic acid microorganisms and thus sustain the production of lactic acid via their metabolic processes. The research and development efforts for reducing the costs associated with production of industrial-grad lactic acid via microorganism fermentation have not included devising cheap methods of using or preserving fresh plant materials.

Disclosed herein is “fresh fermented biomass” produced via fermentation, from which industrial lactic acid can be obtained. Substrate for the fermentations disclosed here can be plants that are directly harvested and not processed into any other substance first. In some embodiments, the substrate for the fermentations can be any plant. In some embodiments, the substrate for the fermentations disclosed herein can be plant leaves, stems, and/or roots. In some embodiments the substrates are terrestrial. In some embodiments the substrates are from aquatic fresh water. In some embodiments, the substrates are from aquatic saltwater. In some embodiments, the plant substrate has a moisture level above 50%. In some embodiments, the substrates are fermented into “fresh fermented biomass” in order to store/preserve the material for follow-on fermentation to completion into industrial grade lactic acid (Double Fermentation Embodiment), or the substrates are fermented completely into “fresh fermented biomass” and can be used to obtain industrial grade lactic acid (Single Fermentation Embodiment).

In some embodiments, the plant can be a herbaceous plant (i.e., lacking a persistent woody stem above ground). In some embodiments, the plants can be cultivated and harvested above-ground agricultural crops, such as maize, corn, sorghum, soybeans, beans, peas, rape, foraging grasses, legume grasses, and the like. In some embodiments, the plant can be a vegetable.

In some embodiments, the plant substrate can be cultivated and harvested above and below ground whole-plant (i.e. root and leaf top) root crops, such as turnips, beets, sugar beets, carrots, rutabagas, daikon radish, white radish, potato roots and potato vines, and the like.

In some embodiments, the substrate can be non-cultivated plants, such as grasses, weeds, tubers, broadleafs, perennials, annuals, and the like. Generally, the plant substrates used can have a moisture content higher than 50% at time of processing. There is no limit to moisture content for this method, as the more moisture, the more successful the described herein.

In some embodiments, the plant substrates for fermentation can be root vegetables. Root vegetables can refer to underground parts of plants that can be eaten as food by animals, including humans. Root vegetables can include true roots (e.g., taproots, tuberous roots) and non-roots (e.g., bulbs, corms, rhizomes, tubers). Root vegetables can have leaves and/or greens.

In some embodiments, the root vegetable can be a taproot. Taproots can include beetroot, burdock, carrot, dandelion, parsley, parsnip, radish, rutabaga, sugar beet, turnip, taro, chicory, and the like.

In some embodiments, the root vegetable can have edible greens. In some embodiments, these root vegetables can include arracacha, beets, carrots, celeriac, daikons, ginger, leeks, onions, radishes, rutabaga, turmeric, turnips, sweet potatoes, and the like.

In some embodiments, the root vegetables can be corms, including for example, Amorphophallus konjac (konjac), Colocasia esculenta (taro), Eleocharis dulcis (Chinese water chestnut), Ensete spp. (enset), Nymphaea spp. (waterlily), Pteridium esculentum, Sagittaria spp. (arrowhead or wapatoo), Typha spp., Xanthosoma spp. (malanga, cocoyam, tannia, yautia and other names), Colocasia antiquorum (eddoe or Japanese potato) and the like.

In some embodiments, the root vegetables can be bulbs, including Allium cepa (onion), Allium sativum (garlic), Camassia quamash (blue camas), Foeniculum vulgare (fennel) and the like.

In some embodiments, the root vegetables can be rhizomes, including Curcuma longa (turmeric), Panax ginseng (ginseng), Arthropodium spp. (rengarenga, vanilla lily, and others), Canna spp. (canna), Cordyline fruticosa (ti), Maranta arundinacea (arrowroot), Nelumbo nucifera (lotus root), Typha spp. (cattail or bulrush), Zingiber officinale (ginger, galangal), and the like.

In some embodiments, the root vegetables can be tuberous stems, including Apios americana (hog potato or groundnut), Cyperus esculentus (tigernut or chufa), Helianthus tuberosus (Jerusalem artichoke or sunchoke), Hemerocallis spp. (daylily), Lathyrus tuberosus (earthnut pea), Oxalis tuberosa (oca or New Zealand yam), Plectranthus edulis and P. esculentus (kembili, dazo, and others), Solanum tuberosum (potato), Stachys affinis (Chinese artichoke or crosne), Tropaeolum tuberosum (mashua or añu), Ullucus tuberosus (ulluku), and the like.

In some embodiments, the root vegetables can include Zamia integrifolia (Florida arrowroot), and the like.

In some embodiments, the root vegetables can be taproots, including Arracacia xanthorrhiza (arracacha), Beta vulgaris (beet and mangelwurzel), Brassica spp. (kohlrabi, rutabaga and turnip), Bunium persicum (black cumin), Burdock (Arctium, family Asteraceae), Carrot (Daucus carota subsp. sativus), Celeriac (Apium graveolens rapaceum), Daikon—the large East Asian white radish (Raphanus sativus var. longipinnatus), Dandelion (Taraxacum) spp., Lepidium meyenii (maca), Microseris lanceolata (murnong or yam daisy), Pachyrhizus spp. (jicama and ahipa), Parsnip (Pastinaca sativa), Petroselinum spp. (parsley root), Radish (Raphanus sativus), Scorzonera hispanica (black salsify), Sium sisarum (skirret), Tragopogon spp. (salsify), Vigna lanceolata (bush carrot or bush potato) and the like.

In some embodiments, the root vegetables can be tuberous roots, including Amorphophallus galbra (yellow lily yam), Conopodium majus (pignut or earthnut), Dioscorea spp. (yams, ube), Dioscorea polystachya (nagaimo, Chinese yam, Korean yam, mountain yam, white ñame), Hornstedtia scottiana (native ginger), Ipomoea batatas (sweet potato), Ipomoea costata (desert yam), Manihot esculenta (cassava or yuca or manioc), Mirabilis expansa (mauka or chago), Psoralea esculenta (breadroot, tipsin, or prairie turnip), Smallanthus sonchifolius (yacón) and the like.

In some embodiments, the fermentation will include fermenting both the root and the leaf, together and simultaneously.

Generally, the plants can be of any variety and can include plants which have no economic or food value and are otherwise considered pests or “weeds”. In some embodiments, the plants can be turnips. In some embodiments, a turnip can be Brassica rapa turnips (purple top turnips).

As stated and demonstrated in this application, any “plant” or non-woody part of a plant (i.e. leaf, stem, root), terrestrial or aquatic, can be used as a raw-material feedstock source for the disclosed methods of lactic acid for industrial purposes. In the Examples herein, purple top turnip root+leaf and leaf-only (Brassica rapa) was used as the feed stock for lactic acid production for several reasons: (1) it is a very fast growing plant, (2) it does not compete, as a carbohydrate or protein source, with more taste-desirable substrates (e.g. wheat, rice, corn, soybeans, alfalfa, etc.), in the food supply, (3) required zero inputs (fertilizer, herbicides, irrigation), due to Alaskan soils and the fact the plant was not being grown as a human or animal food source, where weeds decrease yield-per-acre and overall food quality, (4) is extremely well-adapted to grow in multiple climate and temperature conditions throughout the world, (5) has both high crude protein (for buffering), high calcium (for buffering), low in lignin (lignin is very hard for lactic acid bacteria to break down), high in soluble fiber and pectin (easier to convert to lactic acid), low in starch (very difficult for lactic acid bacteria to metabolize), and high simple sugar content (for lactic acid fermentation), all of which combine to produce large (r) amounts of lactic acid than a normal feed-stock (e.g. corn, alfalfa, etc.) for animal feed silage, (6) has high moisture content, to assist in producing more lactic acid and more nutrient-rich effluent, (7) can be grown with extremely large yields per acre (e.g. up to 15 tons in roots and at least 7 more tons in leaf per acre), (8) the leaf, having large amounts of all necessary nutrients to support lactic acid generating microorganisms, is considered a “waste” material and therefore, has no price-point value; (9) the root and leaf and leaf-only substrates proved over and over again to have a superior capability to generate high concentrations of lactic acid, even without reducing to a slurry, and (10) the leaf of sugar beet, daikon radish, potato vines, etc. are all considered wastes and typically left “on the field”. Their use would not compete with other uses for the roots, which may have commercial value, and their chemical make-up and “freshness” are a positive attribute, when trying to produce lactic acid cheaply and abundantly.

### Processing of Plant Material

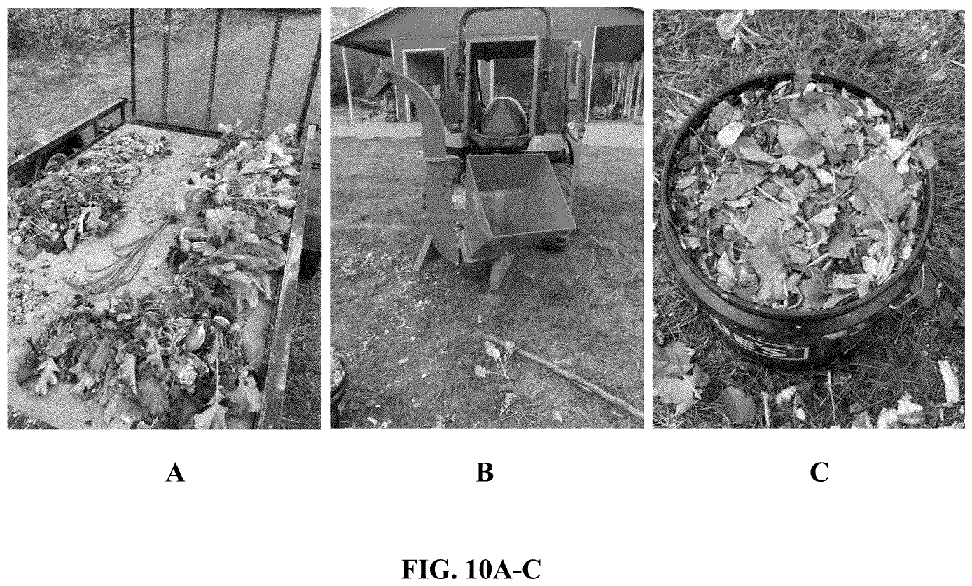

In embodiments where the Double Fermentation Embodiment is used, the raw plant material is first chopped, shredded, and/or chipped and then used in the first fermentation step of the double fermentation process. The fermentation is anaerobic and is accomplished with lactic acid-producing microorganisms. In some embodiments, chopped/shredded/chipped material is illustrated in FIG. 10A-C. In some embodiments, this material has a length of about 2, 3, 4, 5, 6, 7, 8, 9 or 10 inches. Such processing has been described in U.S. Provisional Patent Application Ser. No. 63/566,659, filed Mar. 18, 2024. This ending material from the described feremenation is referred to as “fresh fermented biomass”. The fresh fermented biomass is stored until needed for processing to completion.

In embodiments where the Single Fermentation Embodiment is used, the raw plant material can be blended into a slurry using a combination of steps (e.g. chopped, shredded, chipped, ground, and or blended) or directly slurried and used in a single step of fermentation to completion via lactic acid microorganisms. The slurried biomass, which results from the fermentation is also referred to as “fresh fermented biomass”, as it is chemically identical to the “fresh fermented biomass” which results from the second fermentation step of the Double Fermentation Embodiment. In some embodiments, the plant material is processed to a length of less than about 2, 1, 0.5, 0.4, 0.3, 0.2, 0.1 or less inches in length, or are slurried. This can be done by, for example, grinding/blending the “fresh fermented biomass” via an industrial blender, industrial grinder, or industrial pulverizing machine/device. The fresh fermented biomass is directly fermented to completion.

In embodiments, the fresh, raw plants or plant materials may be inoculated with lactic acid producing microorganisms or inoculated with dissolved sugars or fruit juices to both assist lactic acid fermentation and also direct the type of lactic acid to be produced (e.g. D-lactic or L-lactic acid).

### Fermentation by Lactic Acid Bacteria

Herein, fermentation refers to an anaerobic process, performed by microorganisms (e.g., bacteria). In some embodiments, the process can include breakdown of carbohydrates (e.g., glucose). In some embodiments, energy (e.g., ATP) can be produced. Herein, the source of substrate for the fermentations can be plants, as discussed above. Herein, lactic acid bacteria can perform the fermentations.

In some embodiments, fermentation can be heterofermentative. Herein, heterofermentation can describe a process whereby a microorganism ferments glucose and produces CO₂. In some embodiments, 1 mole of glucose is catabolized to 1 mole of lactic acid, 1 mole of ethanol and 1 mole of CO₂, generating 1 mole of ATP.

In some embodiments, fermentation can be homofermentative. Herein, homofermentation can describe a process whereby a microorganism ferments glucose and does not produce CO₂. In some embodiments, 1 mole of glucose is catabolized to 2 moles of lactic. No CO₂ is produced. Two moles of ATP are generated.

In this disclosure, in some embodiments, at least a part of the fermentation can be performed by homofermentative microorganisms. In some embodiments, a majority of the fermentation can be performed by homofermentative microorganisms. In some embodiments, the fermentation can be exclusively performed by homofermentative microorganisms. The homofermetative microorganisms can be bacteria. In some embodiments, the homofermentative bacteria can be lactic acid bacteria.

Lactic acid bacteria acid can be part of the phylum Firmicutes, class Bacilli, and order Lactobacillus. The order Lactobacillus can include six families, i.e., Aerococcaceae, Carnobacteriaceae, Enterococcaceae, Lactobacilloae, Leuconostocaceae, and Streptococcaceae, over 30 genera, and more than 300 species. Lactic acid bacteria can be divided into two subgroups: heterofermentative lactic acid bacteria and homofermentative LAB.

In some embodiments, homofermentative lactic acid bacteria can be from genera Lactococcus, Enterococcus, Streptococcus, Pediococcus or group I lactobacilli. In some embodiments, the homofermentative bacteria can be Latilactobacillus sakei (formerly genus Lactococcus).

In some embodiments, other lactic acid producing microorganisms are disclosed in Abedi, E., & Hashemi, S. M. B. (2020). Lactic acid production-producing microorganisms and substrates sources-state of art. Heliyon, 6(10).

Herein, plants that are undergoing fermentation, in combination with the microbes facilitating the fermentation, can be referred to as a fermentate. Plants that have completed fermentation (e.g., fermentate that is ready for use as fertilizer) can also be referred to as a fermentate. Fermentates can contain materials that are solid (e.g., plants). Fermentates can also include liquid material, which can be called an effluent. In some embodiments, the fermentate liquid includes lactic acid.

Herein, fermentation can occur under anaerobic or partially anaerobic conditions. In some embodiments, example conditions in which fermentation can occur can be conditions under which obligate anaerobic bacteria can divide and/or ferment carbohydrates. In some embodiments, example conditions in which fermentation can occur can be conditions under which obligate anaerobic and facultative anaerobic bacteria can divide and/or ferment carbohydrates. In some embodiments, example conditions in which fermentation can occur can include up to 2-8% oxygen in the environment.

Plant fermentation, as described herein, can be open fermentation. Open fermentations are open to the environment. For example, making silage (ensiling) can be an open fermentation process. Plants can be piled in a field and remain there for days, weeks or months. Under these conditions, plants on the exterior of the pile are exposed to oxygen (aerobic conditions). Plants on the interior of the pile can be exposed to anaerobic conditions under which fermentation can occur. Open fermentation can occur in vessels, for example, in which the vessel top is removed.

Plant fermentation, as described herein, can be closed fermentation. Closed fermentation can occur in closed, airtight or hermetically-sealed container. Examples of these containers can be closed tanks, fermenters, silos, bunkers, pits, and the like. In some embodiments, a closed container can be a plastic container (e.g., FIG. 10C) onto which an airtight lid can be positioned. In some embodiments, an airtight container that has a valve can be used. In some embodiments, the valve can provide for continuous off-gassing. Other containers can be used. In some embodiments of a closed fermentation, an airtight container can be vented (e.g., during a gas build-up around or soon after—during the first week—the fermentation has been initiated) and then resealed.

Herein, plant material that will undergo fermentation can be exposed to or can be placed under conditions in which fermentation can occur. Conditions under which fermentation can occur can include, for example, a permissive temperature or humidity. In some embodiments, a temperature range for fermentation can be between about 32°-120°Fahrenheit (°F). In some embodiments, a temperature range for fermentation can be greater than about 32 °F and lesser than about 80, 75, 70, 65, 60, 55, 50, 45 or 40 °F. In some embodiments, the ambient air temperature during fermentation can be between about 32°-60 °F.

In some embodiments, the temperature range for fermentation can be the temperature range found in southern Alaska (e.g., Anchorage) during the months of May, June, July, August, September, and October. Conditions under which fermentation can occur can also include presence of a fermentable carbon source, presence of nutrients that provide for the bacteria to grow, water and the like.

Fermentation can take time. In some embodiments, the plant fermentations disclosed herein can last at least 10, 20, 30, 45, 60, 75, 90, 105, 120 or 135 days. In some examples, at the end of this time, the fermentation by the microorganisms to which the plants are exposed can have ceased. In some embodiments, the fermentation can cease because the amount of fermentable material in the fermentation can be low or exhausted, pH of the fermentate may be too low for microorganisms to continue fermenting, and other factors may affect when fermentation ends.

Fermentation requires microorganisms. In some embodiments, plants and/or surfaces of plants naturally contain microorganisms that are capable of fermentation. For example, the bacteria can be naturally available on the plant substrate, in the attached soil or in water naturally adhering to the plant. When the plant materials are exposed to an anaerobic environment under conditions in which fermentation can occur, some of the bacteria associated with the plant can initiate fermentation. This can be the situation when cabbage is fermented to make sauerkraut, for example. In embodiments, the plants used in the methods and products disclosed herein, have associated microorganisms (e.g., bacteria) that can ferment the plant material. In some embodiments, the associated microorganisms can include bacteria. In some embodiments, the associated microorganisms can include lactic acid bacteria. In some embodiments, the lactic acid bacteria can be homofermentative bacteria. In some embodiments, the bacteria can be heterofermentative and homofermentative bacteria.

In some embodiments, an inoculant and/or starter culture of microorganisms (e.g., lactic acid bacteria) can be added to the plant materials at or around the time plant material is exposed to anaerobic conditions. In some embodiments, the inoculant and/or starter cultures can be added to the plant materials after the fermentation has begun. In some embodiments, the inoculant and starter cultures can contain homofermentative lactic acid bacteria. In some embodiments, the inoculants can be bacteria that can produce soluble phosphate and/or potassium from insoluble sources. In some embodiments, dissolved sugar(s) inoculants (e.g. sucrose) can be added to the plant materials at or around the time the plant material is exposed to anaerobic conditions. Adding inoculants, be they starter cultures or dissolved sugars, are meant to initiate dominance of lactic acid bacteria. Additionally, inoculants of starter culture assist in the creation of either D-lactic acid or L-lactic acid, depending upon the starter culture.

The conditions under which the plants are placed can be anaerobic or nearly anaerobic. In some embodiments, the plants can be placed into a confined space that can be enclosed with an airtight lid, door, or covering, in order to prevent the entrance of oxygen into the “pile”. In some embodiments, the plants can be placed into a container or structure where any exposed faces can and are covered by nonpermeable plastic, canvas, tarps, or materials such as soil layers or sand, in order to prevent the entrance of oxygen into the “pile”. In some embodiments, containers like a bucket can be used. These containers can be covered with a lid which generally can be airtight. The lid can be used to seal the container. Additionally, the conditions require non-permeable sides to the confined space to retain all effluent created by the fermentation.

In some embodiments, sugars in the form of molasses or other sucrose, fructose, or glucose-rich substances or concentrates can be added to the plant substrate. This can provide a readily available (initial) source of food for the fermentation. In some embodiments, these additives can selectively promote growth of homofermentative lactic acid bacteria.

Fermentation needs water. In some embodiments, the fresh, raw plants or plant materials (terrestrial, aquatic saltwater, aquatic freshwater) have moisture levels of at least 50% dry matter or greater, with no upper limit to moisture content.

### Double and Single Fermentation Embodiments

In some embodiments, this invention focuses on several improvements to the pretreatment phase of industrial lactic acid production. Recovery of lactic acid relies on the current art of posttreatment methods to obtain, refine and/or process industrial lactic acid. The improvements described herein are enabled via use of a new biomass feedstock (i.e., feedstocks) and methods of using the novel biomass feedstocks in the fermentation to obtain lactic acid (e.g., double fermentation; single fermentation).

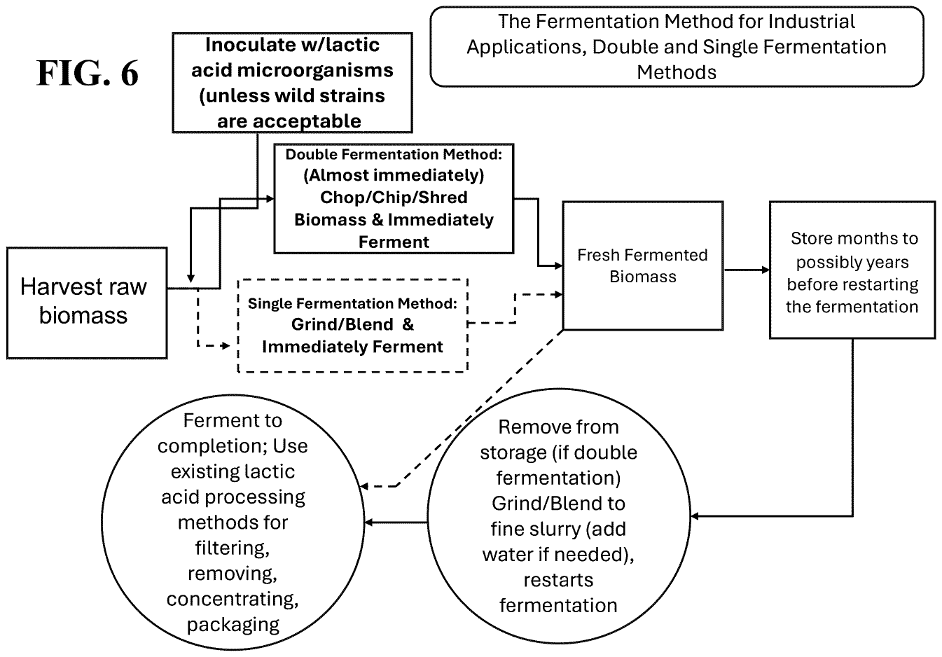

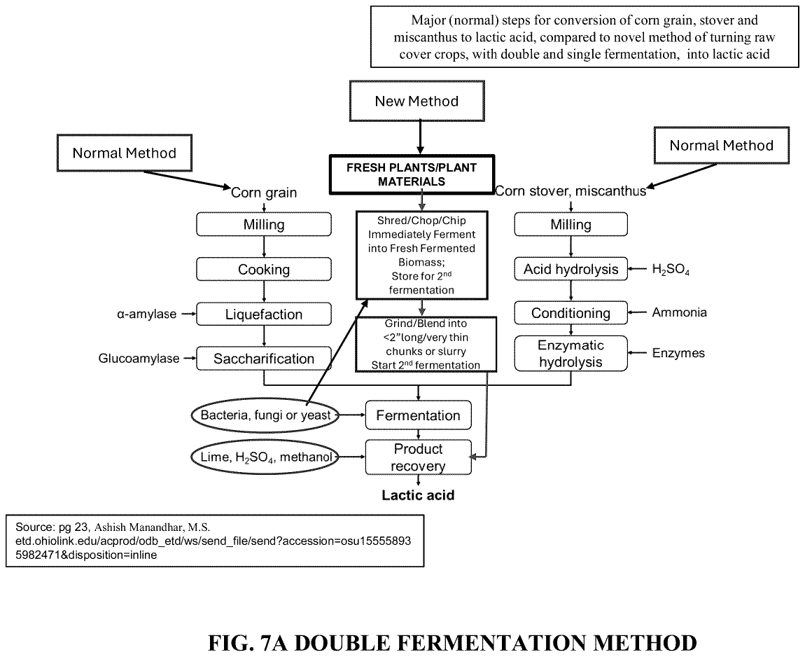

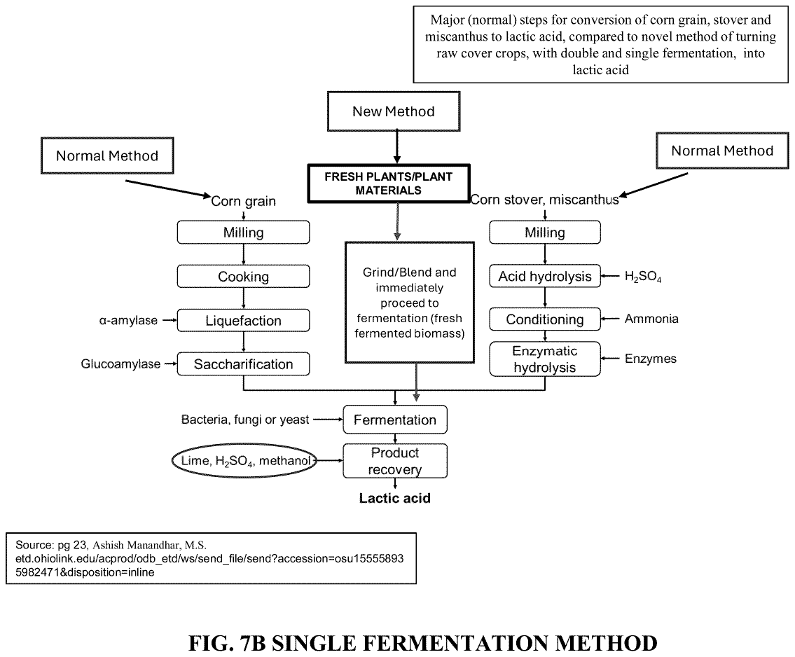

The present invention is in the field of generation of lactic acid by means of fermentation, on an industrial scale, wherein the raw leaf or root/leaf combination of root crops, such as turnip, sugar beet, fodder beet, daikon radish, and the like, or “weeds”, or freshwater or saltwater aquatic plants (herein referred to as “fresh fermented biomass” material) are used as the raw material for fermentation by lactic acid-producing microorganisms (see FIG. 6 for this method, generalized). Utilizing “fresh fermented biomass” material is a new approach to industrial lactic acid. This method is devoted to a process to produce lactic acid by utilizing this biomass feedstock for industrial lactic acid production (see FIGS. 7A and 7B).

In the Double Fermentation Embodiment described herein, the initial fermentation (First Fermentation Step) is stopped/paused at a point prior to complete fermentation. In some embodiments, the fermentation slows or stops without intervention. In some embodiments, the fermentation can slow or stop because microbe-accessible material (e.g., sugars) have been depleted, the temperature is below that at which microbial activity occurs, and/or pH has decreased below a threshold for the bacteria to continue fermentation. At this point in the Double Fermentation Embodiment, the partially fermented fermentate (i.e., microbe-accessible material has been depleted, but microbe non-accessible material can be released by grinding, blending or creating a slurry, for example) is shelf-stable, due to lactic acid microorganism fermentation. This material can later be removed from storage and a follow-on, second lactic acid fermentation (Second Fermentation Step) using a single, mechanical step, and in some embodiments, without the addition of additional nutrients or enzymes, and maintain such fermentation with fewer sustaining inputs (i.e. buffering agents, solubilizing agents, sustaining nutrients, etc.). We refer to this process as the Double Fermentation Embodiment and we consider it a method of producing lactic acid for industrial purposes using the biomass materials described as “fresh fermented biomass”. The slurry can be inoculated with specific types of bacteria which will deliver the end-product results, e.g., as desired by the industrial processing facility (e.g. desired isomer(s) such as D-lactic or L-lactic acid, desired concentrations, desired optical purity, etc.). Once the second fermentation has been re-initiated, the pH will slightly rise but over time, begin to fall as more lactic acid is produced and the buffering effects of the available plant-based protein and/or calcium are exhausted. When this happens, the lactic acid can be removed or additional buffering chemicals such as calcium hydroxide, ammonia, etc. can be added, in order to prolong the fermentation and extract as much lactic acid as possible from the nutrients and sugars remaining in the material. From this point on in the process, the produced lactic acid is refined and concentrated via existing industrial methods.

The Double Fermentation Embodiment can be used when the industrial requirement is for a source of feedstock outside of the normal harvest season. The product/material (i.e. “fresh fermented biomass”) of the First Fermentation Step can be stored until throughput capacity at a lactic acid processing facility is available for follow-on “processing-to-completion” into lactic acid for industrial purposes.

An alternative method is a process by which nutrient rich “fresh fermented biomass” materials are processed directly into industrial-grade lactic acid (i.e., without a second fermentation step). We refer to this alternate method as Single Fermentation Embodiment and the biomass material (also) as “fresh fermented biomass” materials. In the Single Fermentation Embodiment, the plant material is processed to the smaller sizes, as described earlier.

In the Single Fermentation Embodiment, the fresh plants or raw, fresh plant material is immediately ground/blended or slurried and then directly fermented into lactic acid for industrial purposes. Once in the slurry (e.g., in pieces smaller than 2″), it has the attributes of the Double Fermentation Embodiment described above, such as increased surface area, exposing all the trapped sugars, proteins, etc., enabling a more prolonged, unassisted lactic acid fermentation. Identical to Double Fermentation Embodiment, as the fermentation reactions proceed, lactic acid will need to be removed or additional buffering agents (e.g. calcium hydroxide, ammonia, etc.) may need to be added in order to prolong the fermentation and extract as much lactic acid as possible from the remaining material. The lactic acid will be extracted, purified, and processed via existing industrial methods and processes, during this posttreatment phase.

The Single Fermentation Embodiment can be used if the lactic acid processing facility has throughput capacity to immediately accept fresh, raw plants or fresh, raw plant materials. Single fermentation may also be used if the raw plants or raw plant materials are the roots of root crops, which may have been stored over-winter in an unprocessed state/condition. For example, sugar beets are left in piles throughout the winter, awaiting processing into sugar, using the cool conditions to prevent spoilage. When spring arrives with warmer temperatures, single fermentation directly into industrial lactic acid is an option, before the remaining beets spoil. (For clarity, these extra beets could also undergo the double fermentation method, whereby they would be first chopped/chipped/shredded, fermented and then stored indefinitely until ready for the second fermentation). Using aquatic plants/parts of aquatic plants also might be used more in the “Single Fermentation” method than “Double Fermentation” simply because the harvest of these biomass materials (e.g. kelp) do not have the same time constraints as terrestrial plants.

### Feedstocks for Double and Single Fermentation

As used herein, feedstocks can refer to materials that can be used for fermentation to produce lactic acid.

For clarity, “fresh fermented biomass” is a new feedstock option, different from starches (first feedstock option) and lignocellulosic materials (second feedstock option) in the current art.

The products of the Double and Single fermentations described herein are called “Fresh Fermented Biomass” materials and are feedstocks that contain lactic acid. Fresh fermented biomass can be directly processed to obtain/purify lactic acid.

Fresh fermented biomass, in the Double Fermentation Embodiment, is fresh biomass (terrestrial or aquatic plants or plant materials) which has been chopped/chipped/shredded (2″ or greater in size) and has been fermented (via lactic acid producing microorganisms). This material can be stored until a processing facility has throughput capacity and can accept this material and accomplish the second fermentation (via lactic acid producing microorganisms), via the grinding/blending of material into smaller particles (less than 2″) or a slurry.

After the second fermentation, the fresh fermented biomass (Double Fermentation Embodiment) is processed-to-completion into industrial lactic acid, using existing posttreatment methods.

Fresh fermented biomass in the Double Fermentation Embodiment will likely and predominantly come from plants and plant materials which are terrestrial. Terrestrial plant and plant materials, grown within temperate climate zones, usually must be harvested immediately, in-bulk, and made “stable” immediately as they are prone to spoilage. Wet, sweet corn, turnips, and “weeds”, grown within temperate climates, are good examples, as they mature and are harvestable at specific, brief periods in the growing season. They must be harvested and removed from the field quickly and processed quickly (e.g. drying or fermenting or other techniques) to prevent spoilage and waste.

Fresh fermented biomass using the Single Fermentation Embodiment is fresh biomass (terrestrial or aquatic plants or plant materials) which is ground/blended into fine material (less than 2″ or into a slurry) and is fermented (via lactic acid producing microorganisms). The fermented material is processed-to-completion, using existing posttreatment methods, into industrial lactic acid.

Fresh fermented biomass using the Single Fermentation Embodiment will likely and predominantly come from aquatic (freshwater and saltwater) plants and aquatic plant materials. Aquatic plants lend themselves to being harvested throughout a season. Kelp is a good example, as it grows continuously and thus, can be harvested continuously, on-demand, and in quantities that will not overwhelm industrial lactic acid processing facilities.

For clarity, difference(s) between Double and Single fermentation methods is the step in the process whereby raw plants and/or raw plant materials are ground/blended and then processed-to-completion into lactic acid for industrial purposes. In Double Fermentation, raw plants and/or raw plant materials are chopped/chipped/shredded and fermented into fresh fermented biomass and stored. Then, this material is ground/blended for the second fermentation. In Single Fermentation, raw plants and/or raw plant materials are not chopped/chipped/shredded. Instead, the raw plants/plant materials are immediately ground/blended into a fine material and fermented into fresh fermented biomass. There is no storage step and there is no secondary, restarted fermentation step. Fermentation is generally continuous until the fresh fermented biomass is fermented to completion.

### Advantages

Both the Double Fermentation Embodiment and the Single Fermentation Embodiment outlined within this application, when combined with using “fresh fermented biomass”, can result in a method to produce lactic acid for industrial purposes, without the use of the following inputs to initiate and initially sustain fermentation: (1) without the requirement of adding protein, carbohydrate, vitamin, or mineral supplements; (2) without the requirement of adding heat or steam pretreatment(s); (3) without the requirement to add (as much or any) inorganic buffers and inorganic acids; (4) without the requirement to add (as much) water, (5) without the requirement to add enzymes, and (6) without the requirement to add pretreatment acids. This method does not preclude or eliminate the use of the previously stated inputs when the fermentation reactions are “fermented to completion or “processed-to-completion” during the posttreatment phase of industrial lactic acid production, as the use of these inputs may enable the generation of more lactic acid during and throughout the Double Fermentation Embodiment or the Single Fermentation Embodiment. But when performing the Double Fermentation Embodiment, as described herein, the First Fermentation Step is accomplished without any of the previously described inputs whereas the Second Fermentation Step can incorporate these inputs, once and after the second fermentation has been restarted/reinitiated, in order to sustain the second fermentation, beyond the conditions specifically stated and described in our method. The same is true with the Single Fermentation Embodiment, where the need for inputs to initiate and initially sustain fermentations are also not required. The addition of inputs may be necessary to ferment fresh fermented biomass to completion (Single and Double Fermentation Embodiments), but they will be significantly less than the inputs required compared to the other, prior art feedstocks (starches and lignocellulosic feedstocks).

For clarity, in the Double and Single Fermentation Embodiments, no inputs save water and mechanical grinding are required to start (Double and Single Fermentation Embodiments) and/or re-start fermentation (Double Fermentation Embodiment only) by lactic acid microorganisms, but to sustain the biological activity and process-to-completion, buffering or other nutrient inputs may be used.

In part, the disclosed processes and compositions arise from work on fermentation of root vegetables. The findings from the work disclosed herein are that: i) the fermentation of root-crops (leaf or leaf+root) can be accomplished and then restarted at a later date by employing a means to reduce the size of the fermentate such that the material can undergo a second fermentation step; without any requirements for heat, enzymes, acids, buffering agents, or any other inputs or actions, other than the addition of water to aid in the mechanical action of blending/grinding the originally fermented feedstock, and ii) the restarted fermentation of root-crops (leaf or leaf+root) in the previously stated embodiment results in production of additional lactic acid, by percentage dry matter. See FIGS. 7A and 7B for a comparison of the “Double Fermentation” embodiment and the “Single Fermentation” embodiment (generalized) to current methods of using processed starches and lignocellulosic materials.

In the prior art, fermentation has been viewed as an “end”: the step which results in producing industrial lactic acid. The disclosed Double Fermentation embodiment treats fermentation as a “means to an end”, plus an “end”. In the Double Fermentation embodiment, the First Fermentation Step is used first as the technique to preserve and store nutrients and, after the material is in a stable condition, the Second Fermentation step is meant to complete the overall endeavor: produce industrial lactic acid.

It was found that raw turnip leaf and raw turnip leaf/root combinations can undergo a “double fermentation”, without the need for heat, chemicals, or enzyme treatments pretreatments, or the need for buffers and acids during fermentation and post fermentation. Following the steps outlined herein, using previously fermented biomass material, we could re-initiate a lactic acid fermentation, after the previously lactic acid fermented biomass material had been stored for months. The Double Fermentation embodiment used wild strains of both hetero and homofermentative bacteria and generated additional lactic acid, on a dry matter basis, than originally produced in the first fermentation. This experiment generated L-lactic acid. Using exclusively homofermentative bacteria, via inoculants, even higher concentrations of lactic acid can be achieved, as well as different isomers of lactic acid (e.g. D-lactic or L-lactic)

The first fermentation put the bacteria into dormancy for almost 8 months. This is evidenced by the original forage analysis, which measured and identified high levels of preserved proteins and sugars. If the bacteria were not dormant, then these nutrients would have been exhausted.

The second fermentation re-activated the bacteria. This is evidenced by gas buildup and pH changes (lower). Additionally, we cultured live lactic acid bacteria-in very high numbers- and then DNA sequenced these cultures to precisely identify the bacteria. Finally, we measured increased amounts of lactic (by percentage dry matter), compared to earlier fermentation analysis before grinding/blending the material.

These experiments demonstrated “Double Fermentation” harnesses heretofore unutilized biomass feedstocks for the production of industrial lactic acid and provides a method to utilize said biomass feedstocks for the production of industrial lactic acid.

In the “Double Fermentation” method, we take advantage of the small surface area exposed during chopping/chipping/shredding. During this step, the mechanical disruption exposes only a small amount of the raw plant's sugars and proteins to lactic acid producing microorganisms. Thus, in some embodiments, the fermentation activity can be rapid and brief, leaving some or most of the nutrients untouched by bacterial consumption, and in a preserved state. This material, herein called “fresh fermented biomass” can become the feedstock for the Second Fermentation Step of the Double Fermentation method. This fresh fermented biomass material is nutrient-rich, and storable indefinitely, until the factory has throughput capacity to accept this material. Added to this, the nutrient-rich material contains a substantial amount of protein, which was not fermented in the initial fermentation, along with calcium (especially in turnip leaf), which are natural buffering agents.

During the next step of the Double Fermentation Embodiment, the fresh fermented biomass material is ground/blended into a slurry (e.g. w/out needing to add any type of chemical or substance other than water), increasing the surface area of the material and exposing un-fermented nutrients (includes sugars), plant-based protein, and plant-based calcium. The “release” of plant-based protein and/or calcium raises the pH of the original fresh fermented biomass material, allowing dormant lactic acid producing microorganisms to reawaken and begin, once again, to ferment. No nutrients or outside buffering agents or any other processing inputs are required to restart the fermentation, when done in this manner and method. Utilizing the protein (and calcium) in these fresh plants or fresh plant materials (includes aquatic plants/aquatic plant materials) reduces the amount of buffering agent inputs which would (and are) otherwise required in the current art.

Double and Single fermentation methods (1) greatly decrease pre and post processing complexity associated with the current feedstocks by reducing the number of steps and associated equipment, machinery, labor, and energy, (2) greatly decrease the amount of (and sometimes eliminate) inputs, be it chemicals, nutrients, energy, water, etc. required, (3) leverage the protein and calcium within the plant/plant material to self-buffer the fermentation, reducing the amount of chemical buffer inputs, (4) make it now possible and practical to use virtually any fresh, raw plants or raw plant materials, the ideal feedstock but heretofore considered of limited or no practical use (e.g. sugar beet leaf, turnips, daikon radish, weeds, etc.), (5) resolve the storage/spoilage problems associated with using fresh plants/fresh plant materials, which prevented their use and utility in the first place, (6) accommodate the throughput limitations of industrial lactic acid processing facilities, and (7) will not create any additional transportation requirements.

Furthermore, in the method of Double Fermentation, we do not lose any of the original lactic acid produced during the first fermentation step. In fact, additional lactic acid is produced, once the second fermentation begins after the grinding/blending of the material, again, without the need for additional nutrient, buffer, energy, or other chemical inputs. The lactic acid microorganisms reawaken from their dormancy and once again begin to ferment, once the surface area of the fermented biomass has been increased via the grinding/blending.

Another aspect of the production of industrial lactic acid is the overall carbon footprint, or carbon efficiency, of the methods and inputs required to produce lactic acid. The methods required to utilize starches and concentrated sugars and/or the methods required to utilize lignocellulosic materials will have a higher carbon footprint/lower carbon efficiency, than the methods required to utilize fermented raw plants/plant material biomass described within our method(s) of double and/or single fermentation. The carbon footprint for the inputs using starches (such as corn grain) include the cooking, liquefication (using amylase), and saccharification (using glucoamylase). The inputs themselves (such as amylase) require their own energy and inputs to make and must be included within the overall assessment. We will assume the electricity requirement to grind/blend will be somewhat equal between corn grain and our fresh biomass, though this is likely generous, so we will leave this input requirement out, in order to provide a reasonable carbon footprint comparison. All this activity and these inputs are required before the starch is in a state to support fermentation by lactic acid microorganisms. The carbon footprint for using lignocellulosic (such as corn stover or miscanthus) include acid hydrolysis (using sulfuric acid), conditioning (using ammonia), and enzymatic hydrolysis (using enzymes). Again, we will assume the electricity requirement for milling the corn stover/miscanthus is identical to the electricity required to grind/blend the fresh biomass. Again, this is likely a generous comparison. Identical to using starch, all these activities and inputs are required before the lignocellulosic material is in a state to support fermentation by lactic acid microorganisms. This compares to the methods of double and single fermentation, and the use of raw, fresh biomass where the only input requirement is electricity to grind/blend the material. While there are means to do a Life Cycle Analysis, to determine greenhouse gas impacts and compare each of these methods, it is evident without such a high-resolution analysis that the carbon footprint using double and single fermentation, with fresh biomass, will be considerably less than using starches or lignocellulosic materials.

There are other environmental benefits of using single and double fermentation methods, with fresh biomass. Water requirements will be reduced. Depending upon the biomass selected (e.g. turnips, turnip leaves, daikon radish, daikon radish leaves), the amount of water stored within the plants is enough to initiate and sustain, to a point, lactic acid fermentation by microorganisms. Using starches and lignocellulosic materials requires large amounts of water before the materials are in a state where lactic acid fermentation by microorganisms can be initiated.

Waste byproducts such as calcium sulfate (gypsum) are a common and very unfortunate material created when buffers, such as calcium hydroxide, calcium oxide, or other calcium-based chemicals, are used to initiate, sustain, and prolong the lactic acid fermentation process. The buffer reacts with the lactic acid and produces lactate, a salt, which then must be solubilized with sulfuric acid in order to create lactic acid. The unwanted waste byproduct of the lactate+sulfuric acid reaction is gypsum. While there are uses for gypsum, it is a rather common material and the quantities created in this process are immense, adding to the costs (disposal, treatment of, etc.) of the overall process. Because double and single fermentation use the plant protein and calcium to buffer the initial reaction, less amount of outside buffering chemicals are required, thereby generating overall more lactic acid with less waste byproduct. The high amounts of lactic acid generated in both the first fermentation stage of double fermentation, as well as the second fermentation stage of double fermentation, as seen when compared to Table 1 in the Examples, are the result of plant protein buffering and likely, plant calcium buffering as well.

In using fresh fermented biomass, not only are almost all (if not all) pretreatment inputs eliminated in the current art but also some posttreatments inputs as well. During posttreatment, the biomasses, whether they are starches, lignocellulosic biomasses, or fresh fermented biomasses, must be supported with inputs in order to “ferment-to-completion”. In “ferment-to-completion” we mean whereby the fermentation of lactic acid producing microorganisms continues until all available sugars have been metabolized and converted into lactic acid. As has been described earlier, in the use of starches and lignocellulosic biomass materials, buffers, must be continually added in order to support the fermentation and “ferment-to-completion”. Starches and lignocellulosic materials are also deficit in other nutrients such as protein; nutrients which lactic acid producing microorganisms must have in order to metabolize sugars. During fermentation and downstream processes in the current art, nutrients must be continually added in order to compensate for these deficits, thus increasing the overall costs of using these standard biomass materials. In the use of fresh fermented biomasses, fewer buffers are required (as has been described earlier) and also fewer, if any, outside nutrients (such as protein) are required to support the lactic acid microorganisms.

Using starches and lignocellulosic biomass feedstock materials requires a great deal of “brute force” in order to make these materials palatable to lactic acid producing microorganisms. Lactic acid microorganisms have not evolved to use these materials and metabolize them. Fresh biomass, specifically, fresh fermented biomass, on the other hand, coupled with Double and Single Fermentation methods, utilizes biomass materials lactic acid microorganisms have evolved to naturally metabolize. The main purpose of using fresh fermented biomass is to leverage the inherent superiority of fresh materials and their ability to support lactic acid producing microorganisms using as few outside support and inputs as possible, in order to make the overall process more efficient and cost effective.

### Other Considerations

Regarding spoilage of feedstocks: the ideal feedstock for lactic acid fermentation for industrial purposes is a nutrient source which contains all nutrient requirements necessary for the bacteria and is shelf-stable at the same time (i.e. resistant to spoilage). “Food”, whether for human or animal consumption, “spoils” because the nutrients are available and accessible for consumption by microorganisms (i.e. bacteria, yeast, fungi). “Spoilage” occurs when microorganisms start consuming the fresh nutrient source (be it plant or animal). “Food Preservation”, at its core concept and purpose, is simply a technique meant to transform a “fresh/raw” nutrient source from something prone to spoilage by microorganisms into something resistant to spoilage.

Example techniques available to create spoilage resistance are (1) freezing, (2) heating (e.g. pasteurization), (3) salting, (4) radiation, (5) drying/dehydrating, (6) oxygen removal (e.g. vacuum packing), (7) nutrient concentration (e.g. corn starch, granulated sugar, sugar beet juice, etc.) and (8) fermentation via acidity or alcohol. Fermentation uses bacteria to consume the raw nutrient source initially but eventually utilizes the produced acids (or alcohol) to preserve the remaining nutrients. Some acids such as lactic acid (or alcohol) are themselves safe for human (and animal) consumption and simultaneously prevent any other bacteria (as well as the lactic acid bacteria themselves) from consuming to completion the remaining nutrients.

Of the 8 techniques listed previously, the disclosure herein uses #8, fermentation, to resolve any spoilage problem (which arises if biomass is stored for use in a later fermentation). Herein lies the uniqueness of this invention. “Fermentation” has been viewed as an “end” for a single purpose: produce industrial-grade lactic acid. Here, this invention uses fermentation for two purposes: (1) as a “means” to preserve a valuable, easily spoilable substrate and (2) as an “end” to produce industrial-grade lactic acid.

Regarding buffering: Once fermentation has been initiated, whether it be a fermentation of starchy feedstocks or lignocellulosic feedstocks, it must be sustained. Unfortunately, the lactic acid released by the lactic acid bacteria can become self-defeating to the production goal of maximizing lactic acid production by the microbes. As the concentration of lactic acid increases, the acidity also rises (i.e. pH drops). Once the pH drops below a certain point (and this is dependent upon the sensitivities of the lactic acid bacteria selected to ferment the substrate), the lactic acid bacteria may become nonviable or dormant, the fermentation process stops, and the production of lactic acid ceases.

In order to continue the formation of lactic acid, the fermentation broth can be buffered to maintain a high enough pH so that the bacteria can continue to produce lactic acid. In the current art, inorganic buffers are used to maintain the pH, with calcium hydroxide being one of the more common buffers. As the lactic acid is formed, it reacts with the calcium hydroxide or other similar buffers and forms a lactate salt. At the end of the fermentation, the lactate salt can be converted back into lactic acid. The conversion back into lactic acid is predominantly accomplished through the addition of sulfuric acid which, in the case of calcium lactate, produces lactic acid and calcium sulfate (gypsum), a waste product with little utility and expensive disposal costs. The addition of buffers, with the subsequent addition of acids, results in steadily increasing cost and processing complexities and often undesirable and expensive waste byproducts. Using protein as a buffering agent in the production of industrial lactic acid can prevent creation of these waste byproducts. But protein is also valuable as a food source for humans and livestock. Protein can be cost prohibitive and therefore, it is not used in the current art, nor is it considered a viable option to explore via research. However, in the methods disclosed herein, protein is part of the biomass used in the fermentations. This method harnesses unused, discarded protein (as a buffer and nutrient source) that would otherwise be left on the field as a waste (e.g. turnip leaf, sugar beet leaf, “weeds”, etc.).

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Fermenting Root Vegetables

Full turnips (both roots and greens) were harvested. During harvesting, soil was lightly knocked-off of the turnips. The turnips were not washed. The harvested turnips are shown in FIG. 10A.

The harvested turnips-both roots and greens-were shredded using a power take-off wood-chipper (FIG. 10B). The shredded turnips were placed into an HDPE 2 (FDA designates as “Food Grade”) 5-gallon bucket (FIG. 10C). Shredded turnips were compressed by hand into the bucket. After filling to overflowing, the bucket was sealed with an impermeable plastic lid to create an airtight seal to initiate fermentation. Temperature at initiation of fermentation was between 50-60 °F. Nothing was added to the turnips (e.g., additives, inoculants, liquids). Within the first week after fermentation initiation, a slight buildup of gas inside the buckets was evident. The gas buildup was released by slightly opening the edge of the lid and “burping” the gas. After gas release, the lids were immediately closed. The lids remained closed for the duration of the fermentation and opened only at the conclusion of the fermentation, when the fermentate was removed from the buckets. Temperatures during the fermentation were determined using an industrial laser temperature gun, focusing the gun on the external lid top of the buckets. Of note, there were not any temperature spikes, as is usually observed when animal silage processes and methods are used.

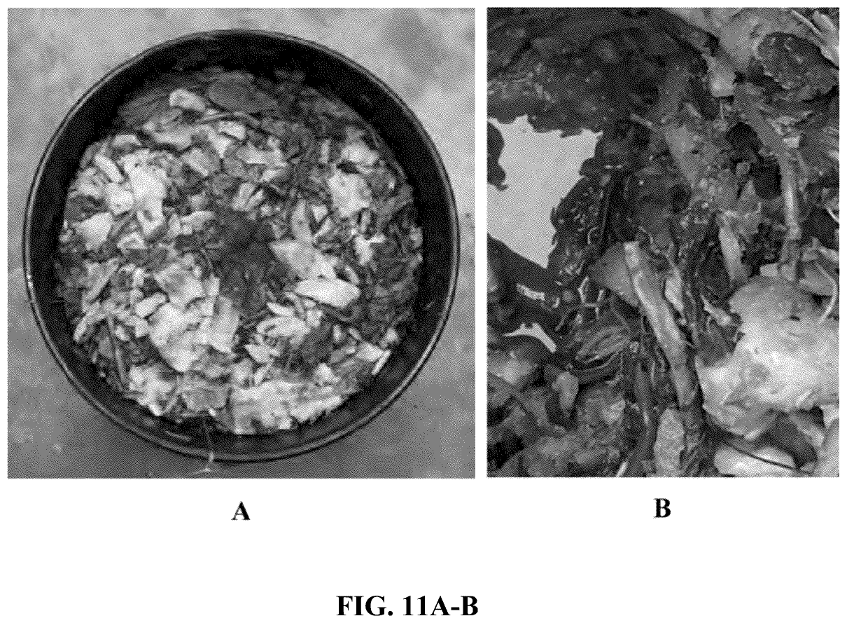

Two turnip fermentations were performed using the methods described above. The first fermentations (Bucket #1 and #2) were sealed on Sep. 10, 2022. The second fermentation (Bucket 3) was sealed on Oct. 2, 2022. The sealed buckets were placed in a root cellar. Temperature in the cellar during the 3-month fermentation period for Buckets #1-3 did not fall below 33 °F Maximum ambient temperature in the root cellar was approximately 59 °F for Buckets #1-2 and approximately 62 °F for Bucket #3. Gas buildup did not appear significant after the initial gas release during the first week of the fermentation, as discussed above. The sealed buckets were then not opened until completion of the fermentation process (about 3 months). Upon unsealing, pH of the fermentate was around 4.0 to 4.4 as measured with a handheld pH meter. Upon unsealing, the fermentate appeared green, was very wet (no effluent was removed during the fermentation) and had a smell of vinegar and/or pickled substances (FIG. 11A-B).

### Example 2—Root Crops (Root-Only, Root+Leaf, Leaf-Only) as a Feedstock for Industrial-Grade Lactic Acid: 2022 and 2023 Lactic Acid Levels Compared to Other Substrates, on a % Dry Matter Basis

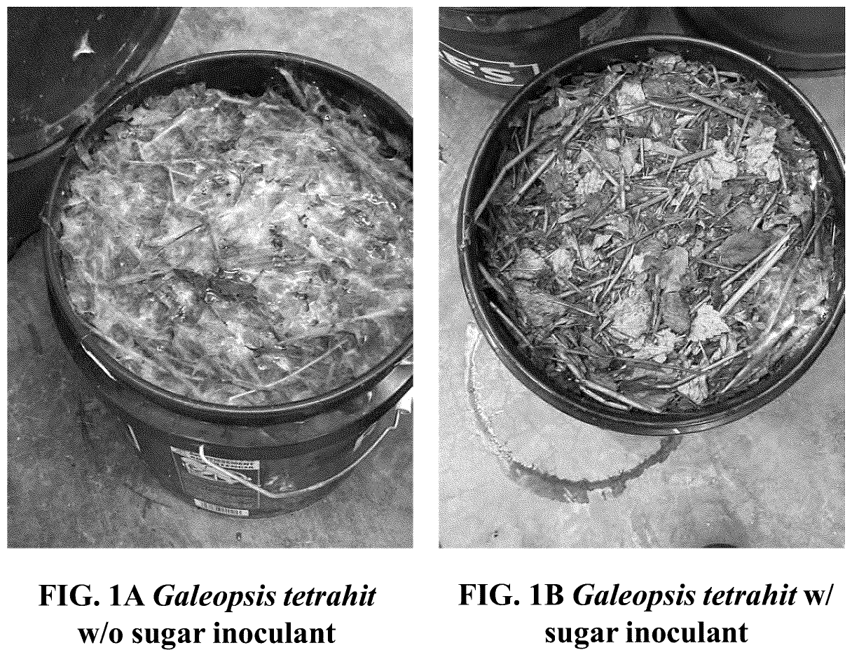

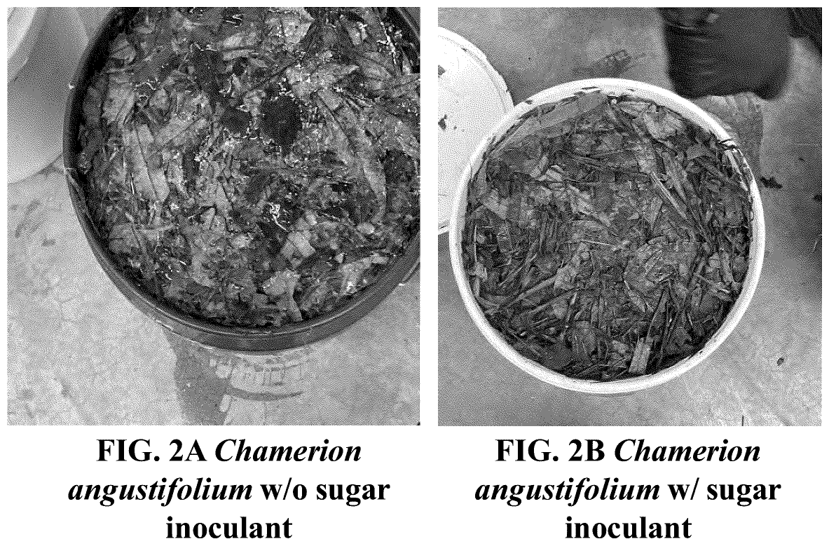

During the summers of 2022 and 2023, we experimented with fermenting various substrates (e.g. turnip leaf, turnip root, turnip leaf and root, hump nettle, and fireweed), at various plant maturities, and different combinations (e.g. root only, leaf only, and root-leaf combinations), using the steps described in FIG. 6. No substrates or their combinations were inoculated with homofermentative bacteria, as we wanted to see if we could get superior results, even with natural wild strains (which the previous year, consisted of both homofermentative and heterofermentative microorganisms). FIG. 1B and FIG. 2B of the “weed” experiments received inoculation of dissolved brown sugar (Galeopsis tetrahit aka “hemp nettle” and Chamerion angustifolium aka “fireweed” inoculated with and without brown sugar and water). Samples were opened and sent at varying times, sometimes immediately after opening and sometimes weeks after opening, to Cumberland Valley Analytical Services (CVAS) for fermentation testing. CVAS utilized a YSI 2900 Biochemistry Analyzer, which detects and measures L-Lactic Acid. CVAS then computed and reported the amount of lactic acid into % dry matter (DM). The results of these 2023 fermentations were combined with fermentation results from growing season 2022, computed on a Dry Matter basis (See Tables 1A and 1B, which contain largely the same data but are formatted differently) using a YSI 2900 Biochemistry Analyzer Cumberland Valley Analytical Services. The fermentation comparisons between samples (chopped/chipped/shredded are 2″ or larger vs ground/blended which are 2″ or smaller) are contained within the shaded boxes (Table 1A), with the shaded boxes on the left highlighting the baseline lactic acid concentration (by % dry matter) compared to the double fermented “ground/blended” of the same original material (by % dry matter) in the shaded boxes on the right.

TABLE 1A

| | | | Turnip | Turnip | T Root | T Root | | | | | | GROUND | GROUND |
|---|---|---|---|---|---|---|---|---|---|---|---|---|---|
| | | | Silage | Silage | Only | Only | T Leaf | Galeopsis | T Leaf | Turnip | Daikon | Turnip | Root + |
| | Turnip | Turnip | #1 | #2 | Dirty | Clean | Only | (Hemp | Only | Silage | Radish | Leaf | Leaf |
| | Silage | Silage | Ensiled | Ensiled | Ensiled | Ensiled | Ensiled | Nettle) | Ensiled | Ensiled | Ensiled | Ensiled | Ensiled |
| | 10 Sep. | 2 Oct. | 15 Aug. | 10 Sep. | 24 Sep. | 24 Sep. | 17 Aug. | 5 Aug. | 17 Aug. | 15 Aug. | 23 Sep. | 17 Aug. | 15 Aug. |
| % DM | 2022 | 2022 | 2023 | 2023 | 2023 | 2023 | 2023 | 2023 | 2023 | 2023 | 2024 | 2023 | 2023 |
| DM | 8.90 | 10.70 | 10.27 | 11.20 | 11.00 | 9.70 | 12.00 | 16.70 | 12.50 | 11.50 | 10.00 | 8.60 | 7.14 |
| pH | 4.76 | 4.11 | 4.41 | 4.69 | 4.54 | 4.31 | 4.64 | 4.60 | 4.60 | 4.35 | 4.72 | 4.46 | 4.12 |
| Ammonia | 3.79 | 1.57 | 3.04 | 1.87 | 0.50 | 0.10 | 1.48 | 4.18 | 15.40 | 13.30 | 1.59 | 1.63 | 2.44 |
| % CP | | | | | | | | | | | | | |
| Total | 14.40 | 9.98 | 14.80 | 5.38 | 5.54 | 7.13 | 14.10 | 4.61 | 15.10 | 19.40 | 24.19 | 17.50 | 21.60 |
| VFA | | | | | | | | | | | | | |
| Lactic | 72.30 | 77.20 | 76.20 | 50.20 | 41.50 | 56.10 | 88.70 | 60.70 | 83.00 | 85.00 | 95.39 | 85.30 | 82.40 |
| Acid as | | | | | | | | | | | | | |
| % Total | | | | | | | | | | | | | |
| VFA | | | | | | | | | | | | | |
| Lactic | 9.60 | 7.70 | 11.30 | 2.70 | 2.30 | 4.00 | 12.50 | 2.80 | 12.30 | 16.40 | 23.07 | 14.70 | 17.80 |
| Acid | | | | | | | | | | | | | |
| Acetic | 3.38 | 2.28 | 3.52 | 2.68 | 3.24 | 3.13 | 1.60 | 1.81 | 2.58 | 2.97 | 1.12 | 2.53 | 3.81 |
| Acid | | | | | | | | | | | | | |
| Propionic | 1.16 | 0.00 | 0.00 | 0.00 | 0.00 | 0.00 | 0.00 | 0.00 | 0.21 | 0.00 | ND | 0.02 | 0.00 |
| Acid | | | | | | | | | | | | | |
| Methanol | NT | NT | 0.55 | 0.57 | 0.27 | 0.30 | 0.45 | 0.18 | 0.42 | 0.62 | 0.42 | 0.41 | 0.77 |
| Ethanol | NT | NT | 4.58 | 3.95 | 4.40 | 3.76 | 5.36 | 2.93 | 3.00 | 3.16 | 0.75 | 4.41 | 10.92 |
| Lactic:Acetic | 2.84 | 3.38 | 3.21 | 1.01 | 0.71 | 1.28 | 7.81 | 1.55 | 4.77 | 5.52 | 20.60 | 5.81 | 4.67 |
| Ratio | | | | | | | | | | | | | |
| Approximate | 55.00 | 77.00 | 75.00 | 101.00 | 115.00 | 115.00 | 77.00 | 65.00 | 77.00 | 75.00 | 53.00 | 77.00 | 75.00 |
| Growth | | | | | | | | | | | | | |
| Days | | | | | | | | | | | | | |
| Fermentation | 94.00 | 106.00 | 105.00 | 93.00 | 79.00 | 65.00 | 103.00 | 115.00 | 228.00 | 230.00 | 162.00 | Double | Double |
| Days Until | | | | | | | | | | | | Fermen- | Fermen- |
| Opening | | | | | | | | | | | | tation | tation |
| | | | | | | | | | | | | Experiment | Experiment |
| Opened | Dec. | Jan. | Nov. | Dec. | Dec. | Nov. | Nov. | Nov. | 1 Apr. | 1 Apr. | 10 Jan. | 1 Apr. | 1 Apr. |
| Date | 13, | 16, | 28, | 12, | 12, | 28, | 28, | 28, | 2024 | 2024 | 2025 | 2024 | 2024 |
| | 2022 | 2023 | 2023 | 2023 | 2023 | 2023 | 2023 | 2023 | | | | | |
| Sampled | Dec. | Jan. | Dec. | Dec. | Dec. | Dec. | Dec. | Nov. | 1 Apr. | 1 Apr. | 10 Jan. | 22 Apr. | 22 Apr. |
| Date | 13, | 16, | 12, | 12, | 12, | 12, | 12, | 28, | 2024 | 2024 | 2025 | 2024 | 2024 |
| | 2022 | 2023 | 2023 | 2023 | 2023 | 2023 | 2023 | 2023 | | | | | |

NT = Not tested
Material used for Double Fermentation Experiment
Material resulting from Double Fermentation Experiment

TABLE 1B

| | | | | | | BASELINE | | | |
|---|---|---|---|---|---|---|---|---|---|
| | | | | | | Turnip | | | |
| | | T Root | | | BASELINE | Silage | GROUND | GROUND | |
| | | Only | T Leaf | Galeopsis | T-Leaf | Ensiled | Turnip | Root + | Daikon |
| | Turnip | Dirty | Only | (Hemp | Only | 15 Aug. | Leaf | Leaf | Radish |
| | Silage | Ensiled | Ensiled | Nettle) | Ensiled | 2023 | Ensiled | Ensiled | Ensiled |
| | 10 Sep. | 24 Sep. | 17 Aug. | 5 Aug. | 17 Aug. | root + | 17 Aug. | 15 Aug. | 23 Sep. |
| % DM | 2022 | 2023 | 2023 | 2023 | 2023 | leaf | 2023 | 2023 | 2024 |
| DM | 8.90 | 11.00 | 12.00 | 16.70 | 12.50 | 11.50 | 8.60 | 7.14 | 10.00 |
| pH | 4.76 | 4.54 | 4.64 | 4.60 | 4.60 | 4.35 | 4.46 | 4.12 | 4.72 |
| Ammonia | 3.79 | 0.50 | 1.48 | 4.18 | 15.40 | 13.30 | 1.63 | 2.44 | 1.59 |
| % CP | | | | | | | | | |
| Total VFA | 14.40 | 5.54 | 14.10 | 4.61 | 15.10 | 19.40 | 17.50 | 21.60 | 24.19 |
| Lactic Acid | 72.30 | 41.50 | 88.70 | 60.70 | 83.00 | 85.00 | 85.30 | 82.40 | 95.39 |
| as % | | | | | | | | | |
| Total VFA | | | | | | | | | |
| Lactic Acid | 9.60 | 2.30 | 12.50 | 2.80 | 12.30 | 16.40 | 14.70 | 17.80 | 23.07 |
| Acetic Acid | 3.38 | 3.24 | 1.60 | 1.81 | 2.58 | 2.97 | 2.53 | 3.81 | 1.12 |
| Propionic | 1.16 | 0.00 | 0.00 | 0.00 | 0.21 | 0.00 | 0.02 | 0.00 | ND |
| Acid | | | | | | | | | |
| Methanol | NT | 0.27 | 0.45 | 0.18 | 0.42 | 0.62 | 0.41 | 0.77 | 0.42 |
| Ethanol | NT | 4.40 | 5.36 | 2.93 | 3.00 | 3.16 | 4.41 | 10.92 | 0.75 |
| Lactic:Acetic | 2.84 | 0.71 | 7.81 | 1.55 | 4.77 | 5.52 | 5.81 | 4.67 | 20.60 |
| Ratio | | | | | | | | | |
| Approximate | 55.00 | 115.00 | 77.00 | 65.00 | 77.00 | 75.00 | 77.00 | 75.00 | 53.00 |
| Growth Days | | | | | | | | | |
| Fermentation | 94.00 | 79.00 | 103.00 | 115.00 | 228 days of | 230 days of | Results | Results | 162.00 |
| Days Until | | | | | Fermentation | Fermentation | from | from | |
| Opening | | | | | Original | Original | Double | Double | |
| | | | | | Material for | Material for | Fermentation | Fermentation | |
| | | | | | Double | Double | Experiment | Experiment | |
| | | | | | Fermentation | Fermentation | | | |
| | | | | | Experiment | Experiment | | | |
| Opened Date | Dec. 13, | Dec. 12, | Nov. 28, | Nov. 28, | 1 Apr. | 1 Apr. | 1 Apr. | 1 Apr. | 10 Jan. |
| | 2022 | 2023 | 2023 | 2023 | 2024 | 2024 | 2024 | 2024 | 2025 |
| Sampled Date | Dec. 13, | Dec. 12, | Dec. 12, | Nov. 28, | 1 Apr. | 1 Apr. | 22 Apr. | 22 Apr. | 10 Jan. |
| | 2022 | 2023 | 2023 | 2023 | 2024 | 2024 | 2024 | 2024 | 2025 |

NT = Not tested

We compared the turnip lactic acid concentrations (as a percentage of dry matter) against other raw substrates commonly used to create silages for animal feed. The following Table 2 is taken from Dairyland Laboratories Inc. It shows the normal and expected lactic acid concentrations (as a percentage of dry matter) from various and common silage substrates.

TABLE 2

| | Legume-Mixed | | | | Corn | |
|---|---|---|---|---|---|---|
| | Legume Silage | | | Grass | Silage | HM Corn |
| Moisture % | 65%+ | 50-65% | <50% | Silage <70% | 60-65% | 25-30% |
| pH | 4.0-4.3 | 4.3-4.7 | 4.7-5.0 | 4.3-4.7 | 3.8-4.2 | 4.0-4.5 |
| Lactic Acid | 6.0-8.0 | 4.0-6.0 | 2.0-4.0 | 6.0-10.0 | 5.0-10.0 | 1.0-2.0 |
| Acetic Acid | 1.0-3.0 | 0.5-2.5 | 0.5-2.0 | 1.0-3.0 | 1.0-3.0 | <0.5 |
| Propionic Acid | <0.5 | <0.25 | <0.1 | <0.1 | <0.1 | <0.1 |
| Butyric Acid | <0.5 | <0.25 | <0.1 | <0.1 | <0.1 | <0.1 |
| Iso-Butyric Acid | <0.1 | <0.1 | <0.1 | <0.1 | <0.1 | <0.1 |
| Ethanol | <1.0 | <1.0 | <0.5 | <1.0 | <3.0 | <2.0 |
| Methanol | <0.5 | <0.5 | <0.5 | <0.5 | <0.5 | <0.5 |
| 2-Butanol | <0.5 | <0.5 | <0.5 | <0.5 | <0.5 | <0.5 |
| 1-Propanol | 0.5-1.5 | 0.5-1.5 | 0.5-1.5 | 0.5-1.5 | 0.5-1.5 | 0.5-1.5 |
| 1,2- | 0-3.0 | 0-3.0 | 0-3.0 | 0-3.0 | 0-3.0 | 0-3.0 |
| Propanediol** | | | | | | |
| Ammonia - CP | <15.0 | <12.0 | <10.0 | <12.0 | <8.0 | <10.0 |
| (% of CP) | | | | | | |
| Lactic:Acetic ratio | 2+ | 2.5+ | 2.5+ | 2+ | 3+ | 3+ |
| Lactic (% of | 60+ | 70+ | 70+ | 60+ | 70+ | 70+ |
| total acids) | | | | | | |

Fermentation Profile Target Levels
Source: Dairyland Laboratories, Inc

The 2023 results duplicated, and in some cases surpassed, the superior lactic acid production levels of growing season 2022. The only cases where this did not occur were with turnip root-only (washed and unwashed) and a root plus leaf combination fermented on 24 Sep. 2023. Without being held to any theory, there are several likely reasons for this: (1) the maturity of the plants was higher than the other fermentations; it is possible the lignin content was higher and thus, fermentation was more difficult, (2) in the root-only fermentations in particular, the roots have a relatively low protein content; protein is a natural buffering agent and without it present in-quantity, the pH of the fermentation likely dropped very quickly and thus stopped the production of additional lactic acid, (3) in the root-only fermentations in particular, the roots have a high concentration of simple sugars that can be readily converted into glucose; high glucose concentrations can have an inhibitory effect on lactic acid bacteria (e.g. glucose toxicity/glucose repression).

The low protein and high sugar content of the root, and its inability to maintain a high pH for a prolonged period of time, has a well understood effect on the duration of a lactic acid fermentation and its correlating effect on the amount of lactic acid which can be produced in other types of plants. Without wishing to be held to any theory, this may be a reason why our method of using the root and leaf, or leaf-only fermentations can achieve higher than normal production of lactic acid, compared with other agricultural substrates. With plenty of simple sugar for the lactic acid bacteria to metabolize, and plenty of protein in the leaf to buffer and “restrict” a sudden collapse in pH (i.e. increase in acidity), the fermentation can be prolonged and produce the higher levels of lactic acid reflected in the results of Table 1. This is evident with the 2024 daikon radish results, with 1-lactic acid generated at 23.07% by dry matter.

### Example 3—A Listing and Description of the Researched and/or Currently Used Feedstocks for Industrial-Grade Lactic Acid Production

There have been many experiments and studies recording achieved concentrations (in g/L) using various homofermentative and heterofermentative bacteria against a myriad of substrate feedstocks, but those feedstocks are exclusively either starch or processed simple sugars (e.g. glucose) or lignocellulosic waste residues from other agricultural processes. Table 3 below, is reproduced from Thomsen, Mette Hedegaard, Jean Pierre Guyot, and Pauli Kiel. “Batch fermentations on synthetic mixed sugar and starch medium with amylolytic lactic acid bacteria.” Applied Microbiology and Biotechnology 74 (2007): 540-546.

Table 3 shows L-Lactate (g/L) results from various starch and synthetic sugar substrates.

TABLE 3
Microbiol Biotechnol (2007) 74: 540-546 545
Table 3 Product formation and substrate consumption in
fermentation of synthetic mixed sugars and starch medium
with amylolytic strains of lactic acid bacteria

| | | | | | | |
|---|---|---|---|---|---|---|
| | | | | | Free sugars: | |
| | | | | | glucose/ | |
| | D- | L- | Total | | fructose/ | |
| Time | Lactate | Lactate | lactate | Acetate | sucrose | Starch |
| (h) | (g/l) | (g/l) | (g/l) | (g/l) | (g/l) | (g/l) |

L. plantarum A6

| | | | | | | |
|---|---|---|---|---|---|---|
| 0 | 0.21 | 0.18 | 0.39 | 4.09 | 3.63/6.52/2.46 | 8.66 |
| 4 | 1.33 | 1.48 | 2.81 | 3.65 | 1.74/5.32/2.35 | — |
| 8 | 4.02 | 4.97 | 8.99 | 3.52 | 0.18/1.51/1.76 | 4.27 |
| 24 | 6.83 | 7.43 | 14.25 | 3.96 | 0.1/0/0 | 3.86 |

L. manihotivorans OND 30

| | | | | | | |
|---|---|---|---|---|---|---|
| 0 | 0.06 | 0.09 | 0.15 | 3.83 | 3.36/6.62/2.71 | 7.94 |
| 6 | 1.37 | 1.71 | 3.07 | 4.05 | 1.34/4.85/2.46 | 9.19 |
| II | 3.92 | 3.95 | 7.87 | 3.7 | 0.05/1.64/2.2 | 6.57 |
| 25 | 5.84 | 5.75 | 11.59 | 5.93 | 0/0/0 | 8.12 |

L. fermentum Ogi E1

| | | | | | | |
|---|---|---|---|---|---|---|
| 0 | 0.36 | 0.31 | 0.66 | 4.05 | 3.26/6.26/2.2 | 8.72 |
| 3 | 0.97 | 1.04 | 2.01 | 5.13 | 1.72/3.3/0 | — |
| 6 | 3.09 | 3.01 | 6.1 | 6.21 | 0/0/0 | 0.955 |
| 9 | 3.31 | 3.36 | 6.66 | 6.38 | 0/0/0 | 0 |

Leuconostoc St3-28

| | | | | | | |
|---|---|---|---|---|---|---|
| 0 | 0.28 | 0.27 | 0.55 | 4.14 | 3.2/6.61/2.63 | — |
| 6 | 2.87 | 2.34 | 5.22 | 3.96 | 0.33/3.41/2.1 | 7.46 |
| II | 4.85 | 6.06 | 10.91 | 3.82 | 0/0/1.82 | 5.85 |
| 25 | 6.8 | 7.58 | 14.38 | 4.57 | 0/0/0.3 | 7.46 |

There have been many experiments recording achieved concentrations, using various homofermentative and heterofermentative bacteria and substrates. Table 4, below, (Abdel-Rahman, Mohamed Ali, Yukihiro Tashiro, and Kenji Sonomoto. “Lactic acid production from lignocellulose-derived sugars using lactic acid bacteria: overview and limits.” Journal of biotechnology 156.4 (2011): 286-301) shows lactic acid (g/L) results from various substrates.

TABLE 4
Lactic acid production from lignocellulosic biomass materials
and lignocellulose-derived sugars by lactic acid bacteria.

| | | | | | P_{LA}^{c} | |
|---|---|---|---|---|---|---|
| | | Ferment. | C_{LA}^{a} | Y_{LA}^{b} | (g L⁻¹ | |
| Microorganism | Substrate | process | (g/L) | (g/g) | h⁻¹) | Reference |
| E. mundtii QU 25 | Cellobiose | Batch | 119 | 0.83 | 1.12 | Abdel-Rahman et al. |
| | | | | | | (2011a) |
| | Xylose | Batch | 86.7 | 0.84 | 0.90 | Abdel-Rahman et al. |
| | | | | | | (2010a, 2011b) |
| | Glucose/ | Batch | 35.1 | 0.91 | 2.99 | Abdel-Rahman et al. |
| | cellobiose | | | | | (2010b, 2011a) |
| | Glucose/ | Batch | — | 0.83 | 3.6^{d} | Abdel-Rahman et al. |
| | xylose | | | | | (2010b) |
| | Glucose/ | Batch | — | 0.79 | 2.6^{d} | Abdel-Rahman et al. |
| | xylose/ | | | | | (2010b) |
| | cellobiose | | | | | |
| E. faecalis RKY1 | Wood | Batch | 93.0 | 0.93 | 1.7 | Wee et al. (2004) |
| | hydrolysate | | | | | |
| E. casseliflavus | Xylose and | Batch | 95.0 | — | — | Taniguchi et al., 2004 |
| and Lb. casei | glucose | | | | | |
| Lb. bifermentans | Wheat bran | Batch with | 62.8 | 0.83 | 1.17 | Givry et al. (2008) |
| DSM 20003 | hydrolysate | cell | | | | |
| Lb. brevis | Corncob | immobilization | 39.1 | 0.70 | 0.81 | Guo et al. (2010) |
| | | Batch | | | | |
| Lb. brevis and Lb. | Wheat straw | Batch | 7.1 | 0.95 | — | Garde et al. (2002) |
| pentosus | hemicellulose | | | | | |
| Lb. casei NCIMB | Cassava | Batch SSF | 83.8 | 0.96 | 1.40 | John et al. (2006a) |
| 3254 | bagasse | | | | | |
| Lb. casei subsp | Soft wood | Batch | 21.1-23.75 | 0.74-0.83 | 0.15-0.23 | Iyer et al. (2000) |
| rhamnosus | | | | | | |
| Lb. coryniformis | Cellulose | SSF | 54.0 | 0.89 | 0.5 | Yanez et al. (2003) |
| ATCC 25600 | | | | | | |
| Lb. coryniformis | Pretreated | Batch SSF | 23.4 | 0.56 | 0.48 | Yanez et al. (2005) |
| spp. torquens | cardboard | | | | | |
| ATCC 25600 | | | | | | |
| Lb. delbreuckii | Alfalfa fibers | SSF | 35.4 | 0.35 | 0.75 | Sreenath et al. (2001) |
| Lb. delbreuckii | Cellulose | SSF | 65.0 | 0.18 | — | lyer and Lee (1999a,b) |
| NRRL-B445 | | | | | | |
| Lb. delbrueckii IFO | Defatted rice | SSF | 28.0 | 0.28 | 0.77 | Tanaka et al. (2006) |
| 3202 | bran | | | | | |
| Lb. delbrueckii | Cellobiose | Batch | 90.0 | 0.90 | 2.25 | Adsul et al. (2007b) |
| mutant Uc-3 | | | | | | |
| | Molasses | Batch | 166 | 0.95 | 4.15 | Dumbrepatil et al. (2008) |
| Lb. delbrueckii | Cassava | Batch SSF | 81.9 | 0.94 | 1.36 | John et al. (2006a) |
| NCIM 2025 | bagasse | | | | | |
| Lb. delbrueckil | Sugar cane | Batch SSF | 67.0 | 0.83 | 0.93 | Adsul et al. (2007a) |
| subsp. delbrueckii | bagasse | | | | | |
| Mutant Uc-3 | | | | | | |
| Lb. delbrueckii | Brewer's | Batch | 35.5 | 0.99 | 0.59 | Mussatto et al. (2008) |
| UFV H2B20 | spent grain | | | | | |
| Lb. delbrueckii | Corn cob | Batch/ | 48.7/44.2 | 0.95/0.92 | 1.01/5.7 | Shen and Xia (2006) |
| ZU-S2 | residue | continuous | | | | |
| Lb. casei and Lb. | Date juice | Batch | 60.3 | — | 3.2^{d} | Nancib et al., 2009 |
| lactis | | | | | | |
| Lb. lactis RM 2-24 | Cellobiose | Batch | 80.0 | 0.8 | 1.66 | Singhvi et al. (2010) |
| | a-Cellulose | SSF | 73.0 | 0.73 | 1.52 | Singhvi et al. (2010) |
| Lb. pentosus | Vine shoots | Batch | 24.0 | 0.76 | 0.51 | Moldes et al. (2006) |
| | Barley bran | Batch | 33.0 | 0.57 | 0.60 | Moldes et al. (2006) |
| | husks | | | | | |
| | hydrolysates | | | | | |
| | Corncob | Batch | 26.0 | 0.53 | 0.34 | Moldes et al. (2006) |
| Lb. pentosus ATCC | Vine-trimming | Batch | | 0.77 | 0.84 | Bustos et al. (2004) |
| 8041 | wastes | | | | | |
| | Corn stover | Fed-batch SSF | 74.8 | 0.65 | — | Zhu et al. (2007) |
| Lb. planlarum | Alfalfa fibers | SSF | 46.4 | 0.46 | 0.64 | Sreenath et al. (2001) |
| Lb. plantarum | — | Batch | 1.47/ | — | — | Okano et al. (2010a) |
| (Recombinant) | Glucan/ | | | | | |
| | cellopentaose/ | | | | | |
| | cellohexaose | | | | | |
| | | | 1.27/ | — | — | |
| | | | 1.27 | — | — | |
| Lb. plantarum | Arabinose | Batch | 38.6 | 0.82 | 3.78^{d} | Okano et al. (2009a) |
| (Recombinant) | | | | | | |
| Lb. plantarum | Xylose | Batch | 41.2 | 0.89 | 1.6^{d} | Okano et al. (2009b) |
| (Recombinant) | | | | | | |
| Lb. rhamnosus | Corn stover | SSF | 20.95 | 0.70 | 0.58 | Cui et al. (2011) |
| and Lb. brevis | | | | | | |
| Lb. rhamnosus | Paper sludge | Batch SSF | 73.0 | 0.97 | 2.9 | Marques et al. (2008) |
| ATCC 7469 | | | | | | |
| Lb. rhamnosus | Apple pomace | Batch | 32.5 | 0.88 | 5.41 | Gullon et al. (2008) |
| ATCC 9595 | | | | | | |
| (CECT288) | | | | | | |
| | Cellulosic | SSP | 39.4 | 0.36 | 0.82 | Romani et al. (2008) |
| | biosludge | | | | | |
| Lactobacillus | Rice and | Batch | 129 | 0.95 | 2.9 | Yun et al. (2004) |
| sp. RKY2 | wheat bran | | | | | |
| Lc. lactis IO-1 | Lignocellulosic | Continuous | 27.0 | 0.9 | 6.7 | Wee and Ryu (2009) |
| | hydrolysates | with | | | | |
| | Xylose | cell- | 33.26 | 0.68 | — | Tanaka et al. (2002) |
| | | recycle | | | | |
| | | Batch | | | | |
| | Sugar cane | Batch | 10.9 | 0.36 | 0.17 | Laopaiboon et al. (2010) |
| | baggase | | | | | |
| Leuconostoc lactis | Hydrolyzed | Batch | 2.3 | — | — | Ohara et al. (2006) |
| SHO-47 and SHO- | xylan | | | | | |
| 54 | (Xylooligo- | | | | | |
| | saccharides) | | | | | |

E, Enterococcus; Lb, Lactobacillus; Lc, Lactococcus; SSF, simultaneous saccharification and fermentation.
^{a}Lactic acid concentration (g/L).
^{b}Yield of lactic acid produced (g) to substrate consumed (g).
^{c}Lactic acid productivity.
^{d}Maximum volumetric productivity.

Tables 2, 3, and 4 demonstrate that the use of root crops (leaf and root or leaf-alone) have not been considered as fresh/raw feedstocks to for use in fermentation or used in practical industrial applications or research testing. Furthermore, these tables also demonstrate the use or study of a fermented feedstock, of any substance, where nutrients have not been extracted for some other product or purpose (e.g. brewers spent grains), re-fermented a second time and used to generate lactic acid for the express purpose for industrial uses, has not been done. We have not found any studies or practices relating to fermentation to preserve a feedstock, then a subsequent, secondary fermentation of that feedstock.

### Example 4—2023 Fermentation Experiments: Restarting Fermentations

On 1 Apr. 2024, a 5-gallon bucket of turnip leaf and root, sealed and fermented on 15 Aug. 2023, was opened, sampled, and sent to Cumberland Valley Analytical Services for complete forage and fermentation analysis. Also on 1 Apr. 2024, a 5-gallon bucket of turnip leaf, sealed and fermented on 17 Aug. 2023, was opened, sampled, and sent to Cumberland Valley Analytical services for a fermentation analysis, only. The buckets had been stored in a 40 ft insulated shipping container during the fall and winter months in Alaska. The temperature inside was not allowed to go below 32 degF. Temperatures did not exceed 40 degF during the winter months. The purpose of this testing was to obtain a baseline lactic acid concentration (by % dry matter) for both substances, and for the turnip leaf+root, a baseline nutritional (forage) analysis as well. The forage analysis for turnip leaf+root, of the 15 Aug. 2023 fermentation (opened, sampled, and submitted for testing to CVAS on 1 Apr. 2024) is shown below in Table 5. The results show high levels of protein, soluble fibers, simple sugars and pectins, and macro and micronutrients. This demonstrates nutrients remained available for additional fermentation, and that any continuing biological activities, which would metabolize and consume those nutrients, had been arrested, and those bacteria either killed or put into dormancy. Table 6 shows the results of nutrient analysis of raw turnip leaf and raw turnip root for comparison to Table 5. As one can observe, the pH drops due to the increasing lactic acid produced by the lactic acid bacteria themselves. When the pH drops to a level the lactic acid bacteria can no longer tolerate, they are arrested and become dormant. Thus, they cease to metabolize the substrate. The presence of lactic acid and the low pH environment it creates prevents not just the lactic acid bacteria from metabolizing the plant material but also prevents other unwanted bacteria, yeast, and fungi from doing the same. Thus, most of the material is preserved leaving most of the original energy, protein, etc. of the raw plant. pH is a control for the first step in the double-fermentation process. As long as a low pH is maintained, which means the integrity of the containment remains intact (i.e. no oxygen infiltration or other disturbance), then the substrate will remain stable (i.e. preserved) and ready for the second step (i.e. the slurrying, blending, grinding to small pieces), which is the restarting of the fermentation).

Table 6 provides a full nutrient analysis of raw turnip leaf and raw turnip root. When Table 6 is compared with the forage analyses of post-fermented turnip root+leaf (see Table 5), one can see how an initial fermentation whereby the plant material is chopped/shredded (not ground into a finer pieces or a slurry) retains a great deal of nutrients, energy, and protein; enough to support an independent (i.e. absent inputs or energy) second (double) fermentation for the production of industrial lactic acid.

TABLE 5
Forage Analysis^{c} of Turnip Root + Leaf Fermentation,
initiated 15 Aug. 2023 Harvest, Opened and Sampled 1 Apr. 2024
Chemistry Analysis Results

| | |
|---|---|
| Moisture | 85.5 |
| Dry Matter | 11.5 |

| Proteins | % SP | % CP | % DM |
|---|---|---|---|
| Crude Protein | | | 14.8 |
| Adjusted Protein | | | 14.8 |
| Soluble Protein | | 66.2 | 9.8 |
| Ammonia (CPE) | 20.0 | 13.3 | 1.97 |
| ADF Protein (ADICP) | | 3.4 | 0.50 |
| NDF Protein (NDICP) | | 4.9 | 0.73 |

| Fiber | % NDF | % DM |
|---|---|---|
| ADF | 121.8 | 23.6 |
| NDF | | 19.4 |
| Lignin | 11.85 | 2.30 |

| | |
|---|---|
| % NFC | % DM |

Carbohydrates

| | | |
|---|---|---|
| Silage Acids | 41.0 | 19.4 |
| Ethanol Soluble CHO (ESC-Sugar) | 19.9 | 9.4 |
| Starch | 9.1 | 4.3 |
| Soluble Fiber | 33.1 | 15.64 |
| Crude Fat | | 4.79 |

Minerals

| | |
|---|---|
| Ash (% DM) | 14.49 |
| Calcium (% DM) | 2.54 |
| Phosphorus (% DM) | 0.50 |
| Magnesium (% DM) | 0.35 |
| Potassium (% DM) | 1.74 |
| Sulfur (% DM) | 0.59 |
| Sodium (% DM) | 0.31 |
| Chloride (% DM) | 0.19 |
| Iron (PPM) | 1673.0 |
| Manganese (PPM) | 173.00 |
| Zinc (PPM) | 65.00 |
| Copper (% DM) | 8.00 |

Fermentation

| | |
|---|---|
| pH | 4.35 |
| Total VFA | 19.4 |
| Lactic Acid (% DM) | 16.40 |
| Lactic Acid as % of Total VFA | 85 |
| Acetic Acid (% DM) | 2.97 |
| Propionic Acid (% DM) | ND |
| Nitrate Ion (% DM) | Not Tested |
| Nitrate-Nitrogen (PPM) | Not Tested |

Energy & Index Calculations

| | |
|---|---|
| TDN (% DM) | 71.3 |
| Net Energy Lactation (Mcal/lb) | 0.74 |
| Net Energy Maintenance (Mcal/lb) | 0.82 |
| Net Energy Gain (Mcal/lb) | 0.53 |
| ME (Mcal/lb) | 1.24 |
| Non-Fiber Carbohydrates (% DM) | 47.2 |
| Non-Structural Carbohydrates, ESC (% DM) | 13.7 |
| DCAD (meq/100gdm) | 15.6 |

^{c}https://www.foragelab.com/Services/Forage-and-Feed/Chemistry/

TABLE 6
Forage Analysis^{c} of Raw Turnip Leaf & Raw Turnip Root
Chemistry Analysis Results

| | % DM |
|---|---|
| Moisture | 89.2a/90.3b |
| Dry Matter | 10.8/9.7 |

Proteins

| | % SP | % CP | % DM |
|---|---|---|---|
| Crude Protein | | | 19.2/11.2 |
| Adjusted Protein | | | 19.2/11.2 |
| Soluble Protein | | 52.5/50.3 | 10.1/5.6 |
| Ammonia (CPE) | | | |
| ADF Protein (ADICP) | | 2.2/5.5 | 0.42/0.62 |
| NDF Protein (NDICP) | | 4.2/6.0 | 0.80/0.67 |

Fiber

| | % NDF | % DM |
|---|---|---|
| ADF | 57.5/89.5 | 10.2/14.3 |
| aNDF | | 17.7/16.0 |
| Lignin | 9.39/15.99 | 1.66/2.55 |

| | | |
|---|---|---|
| Carbohydrates | % NFC | % DM |

Silage Acids

| | | |
|---|---|---|
| Ethanol Soluble CHO (ESC-Sugar) | 38.9/66.2 | 18.8/44.7 |
| Starch | 6.8/3.9 | 3.3/2.7 |
| Soluble Fiber | | |
| Crude Fat | | 2.40/1.72 |

Minerals

| | |
|---|---|
| Ash (% DM) | 13.18/4.25 |
| Calcium (% DM) | 4.03/0.51 |
| Phosphorus (% DM) | 0.54/0.41 |
| Magnesium (% DM) | 0.33/0.12 |
| Potassium (% DM) | 0.91/1.22 |
| Sulfur (% DM) | 0.67/0.47 |
| Sodium (% DM) | 0.24/0.16 |
| Chloride (% DM) | 0.26/0.09 |
| Iron (PPM) | 161.00/74.00 |
| Manganese (PPM) | 196.00/25.00 |
| Zinc (PPM) | 70.00/32.00 |
| Copper (% DM) | 4.00/5.00 |

Energy & Index Calculations

| | |
|---|---|
| TDN (% DM) | 71.2/78.0 |
| Net Energy Lactation (Mcal/lb) | 0.74/0.81 |
| Net Energy Maintenance (Mcal/lb) | 0.82/0.93 |
| Net Energy Gain (Mcal/lb) | 0.53/0.63 |
| ME (Mcal/lb) | 1.23/1.37 |
| Non-Fiber Carbohydrates (% DM) | 48.3/67.5 |
| Non-Structural Carbohydrates, ESC (% DM) | 22.1/47.4 |
| DCAD (meq/100gdm) | −2.3125 |

aData for the raw turnip leaf are before the slash (sampled 21 Aug. 2023, Wasilla, Alaska).
bData for the raw turnip root are after the slash (sampled 21 Aug. 2023, Wasilla, Alaska).
^{c}https://www.foragelab.com/Services/Forage-and-Feed/Chemistry/

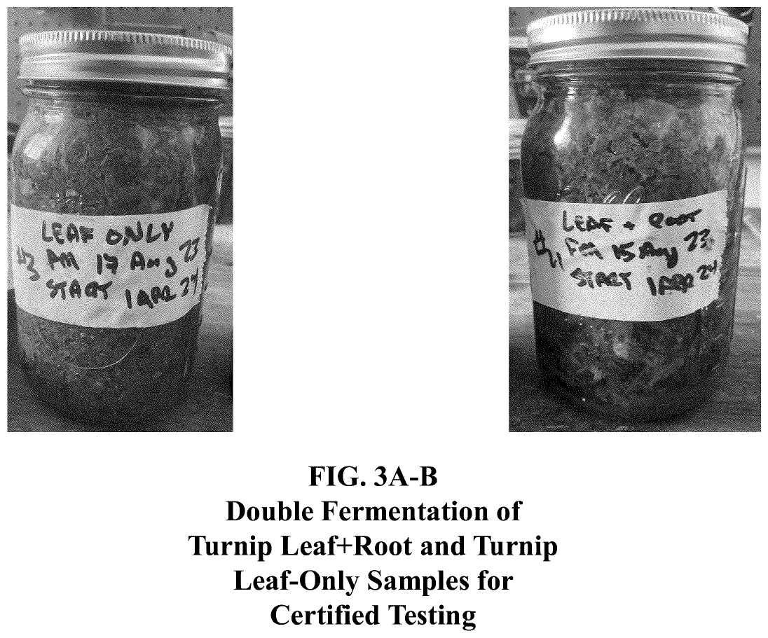

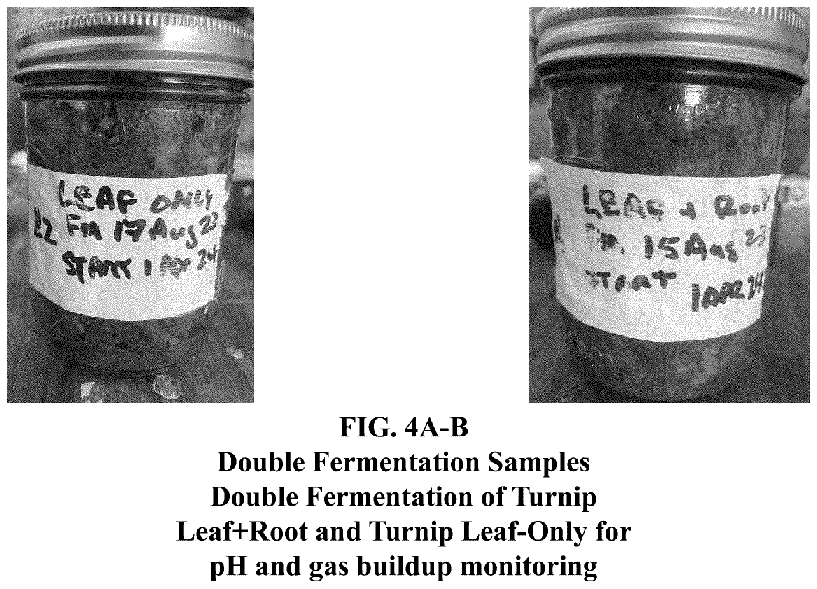

Using some of the remaining material from the same two 5-gallon buckets, samples were taken from each bucket, mixed with tap water, and blended in a food processor into a finer substrate (all accomplished on 1 Apr. 2024). Material of blended leaf and root was put into a 1 quart jar and a 1 pint jar. Material of blended leaf was put into a 1 quart jar and a 1 pint jar (see FIGS. 3A-B and FIGS. 4A-B). Small amounts of tap water were added into the blender, in order to facilitate the blending of the material and also to facilitate lactic acid bacteria metabolism. The ratio of fermented substrate to water was roughly 4 g: 1 mL. The quart jars were periodically “burped” over the next 22 days, by slightly opening the lid and allowing gas to escape before quickly resealing. The pint jars were opened daily for pH testing with a Hanna Instruments handheld pH meter and recorded (see Table 7). Temperatures were the ambient temperatures within the room where the jars were stored (around 62 deg F).

The purpose of this set of testing was to see if fermentation could be restarted from an earlier fermentation, after grinding/blending the substrate into finer particles, thereby increasing the surface area and releasing any carbohydrates and proteins that went unfermented in the initial fermentation. The changes in pH and the gas build-ups, both occurring several days after opening to atmosphere and blending to a finer material, indicated a biological reaction was occurring. Note that the buckets opened on 1 Apr. 2024 did not exhibit any pressure (i.e., there was not a noticeable release of gas when unsealed and opened). This indicates that there was no ongoing gas-releasing biological activity at the time of opening the sealed buckets on 1 Apr. 2024. Furthermore, the forage analysis of Table 5 indicates the biomass material was in a state of preservation.

TABLE 7

| | Root + Leaf | Leaf-Only | Temperature | |
|---|---|---|---|---|
| Date | pH | pH | (ambient) | Comments |
| 1 Apr. 2024 | 4.2 | 4.4 | | |
| 2 Apr. 2024 | 4.3 | 4.3 | | |
| 3 Apr. 2024 | 4.2 | 4.3 | 67 | Gas buildup in Leaf-Only |
| 4 Apr. 2024 | 4.2 | 4.3 | 62 | Gas buildup in Leaf-Only |
| 5 Apr. 2024 | 4.2 | 4.4 | 67 | Gas buildup in Leaf-Only |
| 6 Apr. 2024 | 4.2 | 4.4 | 62 | Slowdown Gas buildup in Leaf-Only |
| 7 Apr. 2024 | 4.2 | 4.4 | 68 | Slight gas buildup |
| 8 Apr. 2024 | 4.2 | 4.4 | 65 | No gas |
| 9 Apr. 2024 | | | | Gas buildup in RootLeaf |
| 10 Apr. 2024 | 4.2 | 4.4 | 62 | Gas buildup in RootLeaf, none in Leaf |
| 11 Apr. 2024 | 4.2 | 4.4 | 64 | Gas buildup in RootLeaf, none in Leaf |
| 12 Apr. 2024 | | | | |
| 13 Apr. 2024 | 4.1 | 4.4 | 62 | No gas either |
| 14 Apr. 2024 | 4.1 | 4.4 | 64 | No gas either |
| 15 Apr. 2024 | 4.1 | 4.4 | | No gas either |
| 16 Apr. 2024 | 4.1 | 4.4 | 65 | Small gas in RootLeaf |
| 17 Apr. 2024 | 4.1 | 4.4 | 65 | |
| 18 Apr. 2024 | 4.1 | 4.4 | 62 | |
| 19 Apr. 2024 | 4.0 | 4.4 | 65 | Small gas in rootleaf |
| 20 Apr. 2024 | 4.0 | 4.4 | 62 | Small gas in rootleaf |
| 21 Apr. 2024 | 4.0 | 4.4 | 67 | Small gas in rootleaf |

The results of the baseline fermentations, restarted fermentations, and a selection of specific earlier test results (fermented root and leaf 15 Aug. 2023 and leaf-only 17 Aug. 2023, sampled 28 Nov. 2023 and tested 12 Dec. 2023) are recorded in Table 1 on a % dry matter basis.

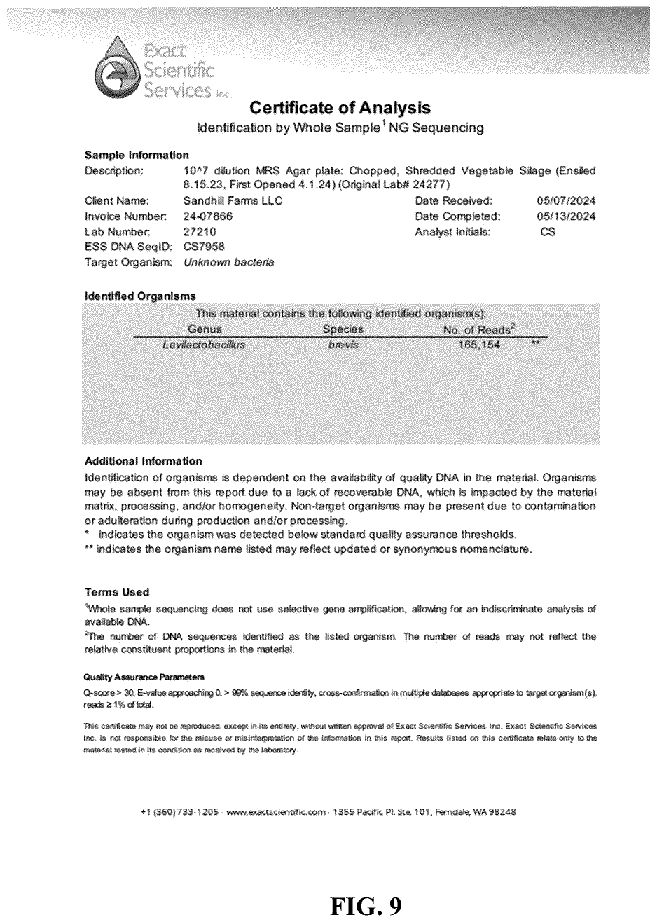

On 22 Apr. 2024, samples of turnip leaf+root and turnip leaf only were sent from the sealed jars in FIGS. 3A and 3B to CVAS for fermentation testing. Those results are recorded in Table 1. Additionally, the sample from FIG. 3B (Turnip leaf+root) was also sent to Exact Scientific Services for 16S DNA sequencing and a probiotic culture count. The results of the 16S DNA sequencing (Table 8) show the top 10 most prevalent microbes in the sample, with the top 3 counts being identified as lactic acid bacteria (Levilactobacillus brevis, Lactiplantibacillus plantarum, and Lactococcus lactis). 16S DNA sequencing does not distinguish between DNA in viable or nonviable microorganisms. To determine whether or not live bacteria were present, a probiotic culture was initiated. The results indicated high amounts (1.4×10\hat{ }8 cfu/g) of an unidentified, live strain of probiotic bacteria (see FIG. 9). A subsequent 16S DNA sequencing of the live culture identified the microorganism as Levilactobacillus brevis and no other bacteria (see FIG. 9).

TABLE 8
Microbes in 16S DNA sequencing of 22 Apr. 2023
Double-Fermented Turnip Leaf + Root sample

| Genus | Species | No. of Reads |
|---|---|---|
| Levilactobacillus | brevis | 258,667 |
| Lactiplantibacillus | plantarum | 55,979 |
| Lactococcus | lactis | 21,175 |
| Kluyvera | intermedia | 10,501 |
| Lelliottia | amnigena | 10,081 |
| Serratia | plymuthica | 8,133 |
| Hafnia | alvei | 2,825 |
| Latilactobacillus | sakei | 1,760 |
| Enerobacter | sp. 638 | 1,344 |
| Raoultella | terrigena | 1,163 |

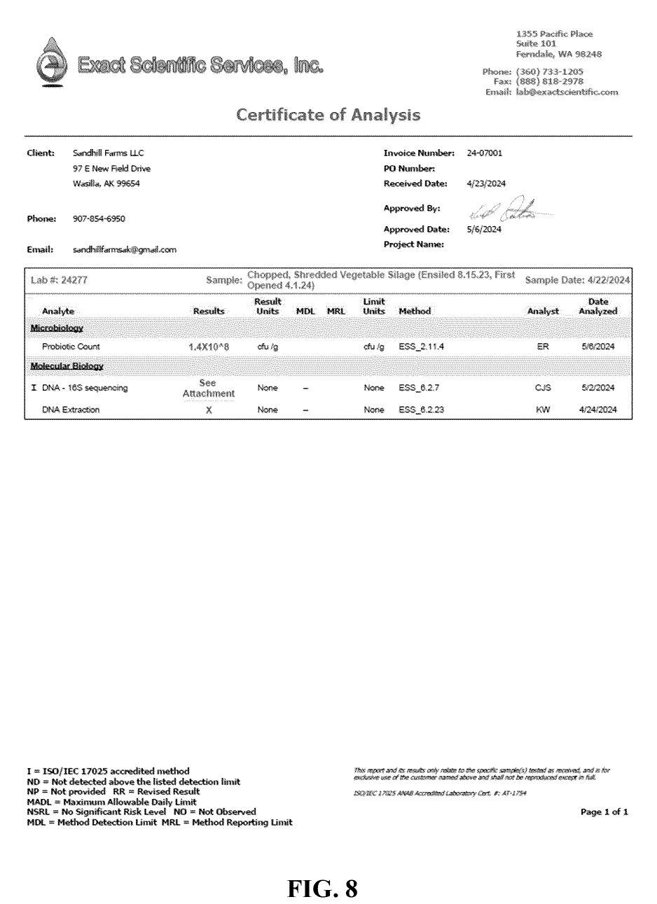

Results: The combination of (1) an intact, preserved nutrient source, high in protein and sugars Table 5, (2) subsequent changes in pH over 22 days of monitoring Table 7, (3) gas buildup days after exposure Table 7, (4) large, live colonies of probiotic bacteria FIG. 8, and (5) subsequent positive identification of live, Levilactobacillus brevis via DNA sequencing of the live probiotic culture(s) FIG. 9, definitively demonstrate a restart of lactic acid fermentation, after months of sealed storage, without the aid of any inputs save a small amount of water and mechanical processing (grinding/blending). Furthermore, increases in lactic acid (on a Dry Matter %) were detected (see Table 1,). Turnip leaf-only increased in lactic acid from 12.30% DM to 14.70% DM, respectively, and turnip leaf+root increased in lactic acid from 16.40% DM to 17.80% DM, respectively.

Analysis and Implications: Lactic acid fermentation can be restarted, after months (~8 months, in this case) in sealed, storage condition, from a previously lactic acid microorganism fermented substance, by simply increasing the surface area of the originally fermented substance, through the mechanical act of grinding and blending. Furthermore, the second fermentation can be initiated and maintained (for at least 22 days) without the requirement to add any buffering chemicals, enzymes, nutrients, vitamins, macro or micronutrients, or energy (heat) and result in increasing amounts of lactic acid (by % dry matter).

The practical application of being able to restart an earlier fermentation is very useful, as it allows for a constant and controlled production of industrial lactic acid year-round. For example, in the production of sugar, sugar beet processing facilities often run in “campaigns”, whereby the sugar beets are only processed for perhaps 3-4 months out of the year, due to sugar beet spoilage. The rest of the year, the processing facility remains shut down.

The practical application of being able to use the fresh parts of plants, which are normally left on the field to rot and decay, is very useful. These parts of plants have no economic value and thus do not compete with other economic endeavors. They are, in fact, an untapped economic resource and, in the case of the art of making lactic acid for industrial purposes, represents an unused biomass substrate. Sugar-beet leaf is an excellent example of a high protein, adequate sugar source, that is left on the field to rot.

In addition to having to rush to process raw beets before spoilage, the processing facility and methods must be able to handle an enormous throughput for a limited amount of time. The product of our First Fermentation Step is able to reinitiate fermentation in the Second Fermentation Step after grinding, blending, and the like.

In our method, the lactic acid producer (e.g. “farmer”) can accomplish an initial fermentation using plants or plant materials (e.g. raw leaf of sugar beet) which currently have limited or zero commercial value or utility and store the fermented biomass until needed by a lactic acid processing facility. Our method proves it can be removed from storage and sent to a processing facility for a second fermentation at the lactic acid processing facility for final processing and purification into industrial lactic acid.

Our method is not constrained to a dual (i.e. Double) fermentation process. If desired, fresh material direct from field can be ground and blended into fine material or slurry and immediately fermented (i.e. Single Fermentation Embodiment). The combination of being able to do double or single fermentations gives great operational flexibility to the production of industrial lactic acid and thereby reduces the overall production costs and enables a more predictable and consistent production of lactic acid for end users. And the ability to use an almost limitless variety of plants or parts of plants, allows for production of lactic acid without interfering with the food supply or other agricultural-based products (e.g. ethanol).

This method provides a stable fermented substrate, ready for a second fermentation to produce additional quantities of industrial lactic acid beyond the first fermentation, without addition of proteins, vitamins, minerals, and the like.

This method uses unique types of raw material substrate(s) (root crop root and leaves or leaf-alone, or “weeds” or aquatic plants). These substrates already contain all the carbohydrates, proteins, minerals, and vitamins necessary to not just allow lactic acid microorganism metabolism, but also to prolong their metabolism (via protein-enabled pH buffering).

This method decreases the production of unwanted waste byproducts, uses less energy, has lower carbon footprint, uses less chemical and nutrient inputs, and uses less water than the methods and materials used in the prior art.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. A fermentation method to produce lactic acid, comprising:
a) chipping, chopping or shredding a plant or plant part into pieces 2″ or larger;
b) fermenting the plant pieces from step (a) with lactic acid bacteria to produce a first fermentate;
c) blending, grinding or slurrying the first fermentate into pieces 2″ or smaller; and
d) fermenting the pieces from step (c) with lactic acid bacteria to produce a second fermentate.

2. The method of claim 1, wherein the plant or plant part used in step (a) has a moisture level above 50% dry matter.

3. The method of claim 1, wherein the fermentation is paused after completion of step (b).

4. The method of claim 1, additionally comprising inoculating the fermenting prior to step (b) and/or prior to step (d) with bacteria capable of producing D-lactic acid and/or L-lactic acid.

5. The fermentation method of claim 1, wherein the plant or plant part comprises roots and leafs/greens.

6. The fermentation method of claim 1, wherein the plant or plant part comprises turnips or daikon radish.

7. The fermentation method of claim 1, wherein the plant or plant part comprises Galeopsis tetrahit or Chamerion angustifolium.

8. The method of claim 1, wherein the lactic acid bacteria comprise Levilactobacillus species.

9. The method of claim 1, wherein the lactic acid bacteria comprise Levilactobacillus brevis.

10. The fermentation method of claim 1, wherein a time between the end of step (b) and the start of step (d) is at least 5, 15 or 30 days, or at least 2, 3, 4, 5, 6, 7, 8, or 9 months.

11. The fermentation method of claim 1, wherein at the end of step (b), not all the plant material has been fermented.

12. The fermentation method of claim 1, additionally comprising a step:
e) extracting lactic acid from the second fermentate.

13. A fermentation method to produce lactic acid, comprising:
a) blending, grinding or slurrying a plant or plant part into pieces 2″ or smaller; and
b) fermenting the plant pieces from step (a) to completion with lactic acid bacteria to produce a fermentate.

14. A material, comprising the first fermentate of claim 1.

15. A material for use in extracting or purifying lactic acid, comprising the second fermentate of claim 1.

16. The material of claim 15, wherein the material is fermented to completion.
